SEQUENCE LISTING (42 sequences)

SEQ ID NO: 1 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 2 - DNA, 1386 bp, Homo sapiens
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 3 - protein, 17 aa, Artificial sequence
  Rigid unit
VAPPPALPAPVRLPGPA

SEQ ID NO: 4 - protein, 18 aa, Artificial sequence
  Rigid unit
VAPPPALPAVAPPPALPA

SEQ ID NO: 5 - protein, 35 aa, Artificial sequence
  Rigid unit
VAPPPALPAVAPPPALPAVAPPPALPAPVRLPGPA

SEQ ID NO: 6 - protein, 35 aa, Artificial sequence
  Rigid unit
VAPPPALPAPVRLPGPAVAPPPALPAVAPPPALPA

SEQ ID NO: 7 - protein, 38 aa, Artificial sequence
  Rigid unit
VAPPPALPAVAPPPALPAGSVAPPPALPAVAPPPALPA

SEQ ID NO: 8 - protein, 42 aa, Artificial sequence
  First linker L1 C1
GGGGSGGGGSGGGGSGGGGSGGGGSVAPPPALPAPVRLPGPA

SEQ ID NO: 9 - protein, 43 aa, Artificial sequence
  First linker L1 C2
GGGGSGGGGSGGGGSGGGGSGGGGSVAPPPALPAVAPPPALPA

SEQ ID NO: 10 - protein, 60 aa, Artificial sequence
  First linker L1 C3
GGGGSGGGGSGGGGSGGGGSGGGGSVAPPPALPAVAPPPALPAVAPPPALPAPVRLPGPA

SEQ ID NO: 11 - protein, 60 aa, Artificial sequence
  First linker L1 C4
GGGGSGGGGSGGGGSGGGGSGGGGSVAPPPALPAPVRLPGPAVAPPPALPAVAPPPALPA

SEQ ID NO: 12 - protein, 63 aa, Artificial sequence
  First linker L1 C5
GGGGSGGGGSGGGGSGGGGSGGGGSVAPPPALPAVAPPPALPAGSVAPPPALPAVAPPPALPA

SEQ ID NO: 13 - protein, 25 aa, Artificial sequence
  First linker L1 GS
GGGGSGGGGSGGGGSGGGGSGGGGS

SEQ ID NO: 14 - protein, 5 aa, Artificial sequence
  Second linker L2
GGGGS

SEQ ID NO: 15 - protein, 10 aa, Artificial sequence
  Second linker L2
GGGGSGGGGS

SEQ ID NO: 16 - protein, 10 aa, Artificial sequence
  Second linker L2
GSGGSGGGGS

SEQ ID NO: 17 - protein, 715 aa, Artificial sequence
  PACE/Furin 1-715
MELRPWLLWVVAATGTLVLLAADAQGQKVFTNTWAVRIPGGPAVANSVARKHGFLNLGQIFGDYYHFWHRGVTKRSLSPHRPRHSRLQREPQVQWLEQQVAKRRTKRDVYQEPTDPKFPQQWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMVALAQNWTTVAPQRKCIIDILTEPKDIGKRLEVRKTVTACLGEPNHITRLEHAQARLTLSYNRRGDLAIHLVSPMGTRSTLLAARPHDYSADGFNDWAFMTTHSWDEDPSGEWVLEIENTSEANNYGTLTKFTLVLYGTAPEGLPVPPESSGCKTLTSSQACVVCEEGFSLHQKSCVQHCPPGFAPQVLDTHYSTENDVETIRASVCAPCHASCATCQGPALTDCLSCPSHASLDPVEQTCSRQSQSSRESPPQQQPPRLPPEVEAGQRLRAGLLPSHLPE

SEQ ID NO: 18 - protein, 746 aa, Artificial sequence
  FIX-C1-Fc-L2-P
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTGGGGSGGGGSGGGGSGGGGSGGGGSVAPPPALPAPVRLPGPADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGSGGGGSLPETGG

SEQ ID NO: 19 - DNA, 2241 bp, Artificial sequence
  FIX-C1-Fc-L2-P
atgcagcgcgtcaatatgattatggccgagtctccaggcctcattaccatttgtctgctgggctacctgctgtccgcagagtgtaccgtcttcctcgaccacgagaacgcaaataagattctcaataggcctaagcgctacaattccggaaagctcgaagagtttgtccagggaaatctggagcgcgagtgtatggaggaaaagtgtagctttgaagaagctagagaggtgtttgagaatactgagcgcactactgagttttggaagcagtatgtggatggcgatcagtgcgagtccaatccatgcctgaatggcggatcttgcaaggatgatattaattcttacgagtgttggtgcccattcggctttgagggaaagaattgcgaactggatgtcacatgtaacatcaagaacggcagatgcgagcagttttgtaagaacagcgccgataataaggtggtgtgtagttgtactgagggataccgcctcgcagagaatcagaagtcctgtgagcccgctgtgccatttccttgtggccgggtcagcgtcagccagacttctaagctgacacgcgccgagaccgtctttccagatgtggattatgtcaactctactgaggccgagaccattctcgataatattactcagtctacacagtcctttaacgacttcactcgggtggtgggcggagaggacgcaaagcctggccagtttccttggcaggtcgtgctcaacggaaaggtggacgcattttgtggcggatctatcgtgaatgagaagtggattgtgaccgccgctcactgcgtcgagacaggcgtcaagattaccgtggtcgctggagagcacaacattgaggaaacagagcacaccgagcagaagcgcaatgtgattcgcatcatcccacaccacaactacaacgccgctattaataagtacaaccacgacatcgccctgttagaactagatgagcctctggtcctgaatagttacgtcactcctatttgtatcgccgataaagaatacactaacatctttctcaagttcggctctggctatgtatctggatggggacgggtgtttcacaagggacggtccgcactcgtgctacaatacctccgggtgccactggtggatcgggcaacatgcctccggtctaccaagtttactatatacaataacatgttttgcgccggctttcacgagggcggacgggatagttgccagggcgatagtggaggccctcatgtcacagaggtggagggaactagctttctgacaggcattatctcttggggcgaagagtgcgctatgaagggaaagtacggcatctatactaaggtctcgcgttacgtcaattggatcaaggaaaagacaaagctgacaggcggcggagggagcggcggcggaggctctggcggcggcggatctggcggaggcggctctggcggaggcggatccgtcgctccaccacctgcactccccgcccctgtcagactgcctggacccgctgataagacccacacatgtccaccttgcccagcacctgagctgctgggcggacctagcgtgtttctgttcccaccaaagccaaaggacaccctcatgattagtagaaccccagaggtgacttgcgtcgtggtcgatgtcagtcacgaggaccccgaggtgaagtttaattggtatgtagatggcgtcgaggtgcacaacgctaagaccaagccacgcgaagagcagtacgcatctacttaccgggtcgtctctgtgctgactgtgctgcaccaggattggctcaatggcaaagagtacaagtgtaaggtcagtaataaggccctccccgcacctattgaaaagactattagcaaggcaaagggccagcctagggagccccaggtctacaccctgcccccttcccgcgatgagctgactaagaatcaggtctctctcacttgcctcgtgaagggcttttacccatccgacattgccgtggagtgggagtccaacggacagccagagaataactacaagacaactcctcccgtgctcgatagcgacggttcattcttcctgtactccaagctcaccgttgataagtctcggtggcagcagggaaatgtgttctcttgttccgtcatgcacgaggcactgcacaatcactacacccagaagtccctgtccctcagccctggcaaaggcagcggcggatctggaggcggcggatccctcccagagaccggcggatga

SEQ ID NO: 20 - protein, 746 aa, Artificial sequence
  FIX-C2-Fc-L2-P
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTGGGGSGGGGSGGGGSGGGGSGGGGSVAPPPALPAVAPPPALPADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGGGSLPETGG

SEQ ID NO: 21 - DNA, 2241 bp, Artificial sequence
  FIX-C2-Fc-L2-P
atgcagcgcgtcaatatgattatggccgagtctccaggcctcattaccatttgtctgctgggctacctgctgtccgcagagtgtaccgtcttcctcgaccacgagaacgcaaataagattctcaataggcctaagcgctacaattccggaaagctcgaagagtttgtccagggaaatctggagcgcgagtgtatggaggaaaagtgtagctttgaagaagctagagaggtgtttgagaatactgagcgcactactgagttttggaagcagtatgtggatggcgatcagtgcgagtccaatccatgcctgaatggcggatcttgcaaggatgatattaattcttacgagtgttggtgcccattcggctttgagggaaagaattgcgaactggatgtcacatgtaacatcaagaacggcagatgcgagcagttttgtaagaacagcgccgataataaggtggtgtgtagttgtactgagggataccgcctcgcagagaatcagaagtcctgtgagcccgctgtgccatttccttgtggccgggtcagcgtcagccagacttctaagctgacacgcgccgagaccgtctttccagatgtggattatgtcaactctactgaggccgagaccattctcgataatattactcagtctacacagtcctttaacgacttcactcgggtggtgggcggagaggacgcaaagcctggccagtttccttggcaggtcgtgctcaacggaaaggtggacgcattttgtggcggatctatcgtgaatgagaagtggattgtgaccgccgctcactgcgtcgagacaggcgtcaagattaccgtggtcgctggagagcacaacattgaggaaacagagcacaccgagcagaagcgcaatgtgattcgcatcatcccacaccacaactacaacgccgctattaataagtacaaccacgacatcgccctgttagaactagatgagcctctggtcctgaatagttacgtcactcctatttgtatcgccgataaagaatacactaacatctttctcaagttcggctctggctatgtatctggatggggacgggtgtttcacaagggacggtccgcactcgtgctacaatacctccgggtgccactggtggatcgggcaacatgcctccggtctaccaagtttactatatacaataacatgttttgcgccggctttcacgagggcggacgggatagttgccagggcgatagtggaggccctcatgtcacagaggtggagggaactagctttctgacaggcattatctcttggggcgaagagtgcgctatgaagggaaagtacggcatctatactaaggtctcgcgttacgtcaattggatcaaggaaaagacaaagctgacaggcggcggagggagcggcggcggaggctctggcggcggcggatctggcggaggcggctctggcggaggcggatccgtcgctccaccacctgcactccccgccgtggctcctccacctgctttgcccgctgataagacccacacatgtccaccttgcccagcacctgagctgctgggcggacctagcgtgtttctgttcccaccaaagccaaaggacaccctcatgattagtagaaccccagaggtgacttgcgtcgtggtcgatgtcagtcacgaggaccccgaggtgaagtttaattggtatgtagatggcgtcgaggtgcacaacgctaagaccaagccacgcgaagagcagtacgcatctacttaccgggtcgtctctgtgctgactgtgctgcaccaggattggctcaatggcaaagagtacaagtgtaaggtcagtaataaggccctccccgcacctattgaaaagactattagcaaggcaaagggccagcctagggagccccaggtctacaccctgcccccttcccgcgatgagctgactaagaatcaggtctctctcacttgcctcgtgaagggcttttacccatccgacattgccgtggagtgggagtccaacggacagccagagaataactacaagacaactcctcccgtgctcgatagcgacggttcattcttcctgtactccaagctcaccgttgataagtctcggtggcagcagggaaatgtgttctcttgttccgtcatgcacgaggcactgcacaatcactacacccagaagtccctgtccctcagccctggcggcagcggcggatctggaggcggcggatctctcccagagaccggcggataa

SEQ ID NO: 22 - protein, 764 aa, Artificial sequence
  FIX-C3-Fc-L2-P
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTGGGGSGGGGSGGGGSGGGGSGGGGSVAPPPALPAVAPPPALPAVAPPPALPAPVRLPGPADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGSGGGGSLPETGG

SEQ ID NO: 23 - DNA, 2295 bp, Artificial sequence
  FIX-C3-Fc-L2-P
atgcagcgcgtcaatatgattatggccgagtctccaggcctcattaccatttgtctgctgggctacctgctgtccgcagagtgtaccgtcttcctcgaccacgagaacgcaaataagattctcaataggcctaagcgctacaattccggaaagctcgaagagtttgtccagggaaatctggagcgcgagtgtatggaggaaaagtgtagctttgaagaagctagagaggtgtttgagaatactgagcgcactactgagttttggaagcagtatgtggatggcgatcagtgcgagtccaatccatgcctgaatggcggatcttgcaaggatgatattaattcttacgagtgttggtgcccattcggctttgagggaaagaattgcgaactggatgtcacatgtaacatcaagaacggcagatgcgagcagttttgtaagaacagcgccgataataaggtggtgtgtagttgtactgagggataccgcctcgcagagaatcagaagtcctgtgagcccgctgtgccatttccttgtggccgggtcagcgtcagccagacttctaagctgacacgcgccgagaccgtctttccagatgtggattatgtcaactctactgaggccgagaccattctcgataatattactcagtctacacagtcctttaacgacttcactcgggtggtgggcggagaggacgcaaagcctggccagtttccttggcaggtcgtgctcaacggaaaggtggacgcattttgtggcggatctatcgtgaatgagaagtggattgtgaccgccgctcactgcgtcgagacaggcgtcaagattaccgtggtcgctggagagcacaacattgaggaaacagagcacaccgagcagaagcgcaatgtgattcgcatcatcccacaccacaactacaacgccgctattaataagtacaaccacgacatcgccctgttagaactagatgagcctctggtcctgaatagttacgtcactcctatttgtatcgccgataaagaatacactaacatctttctcaagttcggctctggctatgtatctggatggggacgggtgtttcacaagggacggtccgcactcgtgctacaatacctccgggtgccactggtggatcgggcaacatgcctccggtctaccaagtttactatatacaataacatgttttgcgccggctttcacgagggcggacgggatagttgccagggcgatagtggaggccctcatgtcacagaggtggagggaactagctttctgacaggcattatctcttggggcgaagagtgcgctatgaagggaaagtacggcatctatactaaggtctcgcgttacgtcaattggatcaaggaaaagacaaagctgacaggcggcggagggagcggcggcggaggctctggcggcggcggatctggcggaggcggctctggcggaggcggatctgtcgctccaccacctgcactccccgccgtggctcctccacctgctttgcccgctgtcgctccaccacctgcactccccgcccctgtcagactgcctggacccgctgataagacccacacatgtccaccttgcccagcacctgagctgctgggcggacctagcgtgtttctgttcccaccaaagccaaaggacaccctcatgattagtagaaccccagaggtgacttgcgtcgtggtcgatgtcagtcacgaggaccccgaggtgaagtttaattggtatgtagatggcgtcgaggtgcacaacgctaagaccaagccacgcgaagagcagtacgcatctacttaccgggtcgtctctgtgctgactgtgctgcaccaggattggctcaatggcaaagagtacaagtgtaaggtcagtaataaggccctccccgcacctattgaaaagactattagcaaggcaaagggccagcctagggagccccaggtctacaccctgcccccttcccgcgatgagctgactaagaatcaggtctctctcacttgcctcgtgaagggcttttacccatccgacattgccgtggagtgggagtccaacggacagccagagaataactacaagacaactcctcccgtgctcgatagcgacggttcattcttcctgtactccaagctcaccgttgataagtctcggtggcagcagggaaatgtgttctcttgttccgtcatgcacgaggcactgcacaatcactacacccagaagtccctgtccctcagccctggcaaaggcagcggcggatctggaggcggcggatccctcccagagaccggcggatga

SEQ ID NO: 24 - protein, 763 aa, Artificial sequence
  FIX-C4-Fc-L2-P
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTGGGGSGGGGSGGGGSGGGGSGGGGSVAPPPALPAPVRLPGPAVAPPPALPAVAPPPALPADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGGGSLPETGG

SEQ ID NO: 25 - DNA, 2292 bp, Artificial sequence
  FIX-C4-Fc-L2-P
atgcagcgcgtcaatatgattatggccgagtctccaggcctcattaccatttgtctgctgggctacctgctgtccgcagagtgtaccgtcttcctcgaccacgagaacgcaaataagattctcaataggcctaagcgctacaattccggaaagctcgaagagtttgtccagggaaatctggagcgcgagtgtatggaggaaaagtgtagctttgaagaagctagagaggtgtttgagaatactgagcgcactactgagttttggaagcagtatgtggatggcgatcagtgcgagtccaatccatgcctgaatggcggatcttgcaaggatgatattaattcttacgagtgttggtgcccattcggctttgagggaaagaattgcgaactggatgtcacatgtaacatcaagaacggcagatgcgagcagttttgtaagaacagcgccgataataaggtggtgtgtagttgtactgagggataccgcctcgcagagaatcagaagtcctgtgagcccgctgtgccatttccttgtggccgggtcagcgtcagccagacttctaagctgacacgcgccgagaccgtctttccagatgtggattatgtcaactctactgaggccgagaccattctcgataatattactcagtctacacagtcctttaacgacttcactcgggtggtgggcggagaggacgcaaagcctggccagtttccttggcaggtcgtgctcaacggaaaggtggacgcattttgtggcggatctatcgtgaatgagaagtggattgtgaccgccgctcactgcgtcgagacaggcgtcaagattaccgtggtcgctggagagcacaacattgaggaaacagagcacaccgagcagaagcgcaatgtgattcgcatcatcccacaccacaactacaacgccgctattaataagtacaaccacgacatcgccctgttagaactagatgagcctctggtcctgaatagttacgtcactcctatttgtatcgccgataaagaatacactaacatctttctcaagttcggctctggctatgtatctggatggggacgggtgtttcacaagggacggtccgcactcgtgctacaatacctccgggtgccactggtggatcgggcaacatgcctccggtctaccaagtttactatatacaataacatgttttgcgccggctttcacgagggcggacgggatagttgccagggcgatagtggaggccctcatgtcacagaggtggagggaactagctttctgacaggcattatctcttggggcgaagagtgcgctatgaagggaaagtacggcatctatactaaggtctcgcgttacgtcaattggatcaaggaaaagacaaagctgacaggcggcggagggagcggcggcggaggctctggcggcggcggatctggcggaggcggctctggcggaggcggatctgtcgctccaccacctgcactccccgcccctgtcagactgcctggacccgctgtcgctccaccacctgcactccccgccgtggctcctccacctgctttgcccgctgataagacccacacatgtccaccttgcccagcacctgagctgctgggcggacctagcgtgtttctgttcccaccaaagccaaaggacaccctcatgattagtagaaccccagaggtgacttgcgtcgtggtcgatgtcagtcacgaggaccccgaggtgaagtttaattggtatgtagatggcgtcgaggtgcacaacgctaagaccaagccacgcgaagagcagtacgcatctacttaccgggtcgtctctgtgctgactgtgctgcaccaggattggctcaatggcaaagagtacaagtgtaaggtcagtaataaggccctccccgcacctattgaaaagactattagcaaggcaaagggccagcctagggagccccaggtctacaccctgcccccttcccgcgatgagctgactaagaatcaggtctctctcacttgcctcgtgaagggcttttacccatccgacattgccgtggagtgggagtccaacggacagccagagaataactacaagacaactcctcccgtgctcgatagcgacggttcattcttcctgtactccaagctcaccgttgataagtctcggtggcagcagggaaatgtgttctcttgttccgtcatgcacgaggcactgcacaatcactacacccagaagtccctgtccctcagccctggcggcagcggcggatctggaggcggcggatctctcccagagaccggcggatga

SEQ ID NO: 26 - protein, 766 aa, Artificial sequence
  FIX-C5-Fc-L2-P
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTGGGGSGGGGSGGGGSGGGGSGGGGSVAPPPALPAVAPPPALPAGSVAPPPALPAVAPPPALPADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGGGSLPETGG

SEQ ID NO: 27 - DNA, 2301 bp, Artificial sequence
  FIX-C5-Fc-L2-P
atgcagcgcgtcaatatgattatggccgagtctccaggcctcattaccatttgtctgctgggctacctgctgtccgcagagtgtaccgtcttcctcgaccacgagaacgcaaataagattctcaataggcctaagcgctacaattccggaaagctcgaagagtttgtccagggaaatctggagcgcgagtgtatggaggaaaagtgtagctttgaagaagctagagaggtgtttgagaatactgagcgcactactgagttttggaagcagtatgtggatggcgatcagtgcgagtccaatccatgcctgaatggcggatcttgcaaggatgatattaattcttacgagtgttggtgcccattcggctttgagggaaagaattgcgaactggatgtcacatgtaacatcaagaacggcagatgcgagcagttttgtaagaacagcgccgataataaggtggtgtgtagttgtactgagggataccgcctcgcagagaatcagaagtcctgtgagcccgctgtgccatttccttgtggccgggtcagcgtcagccagacttctaagctgacacgcgccgagaccgtctttccagatgtggattatgtcaactctactgaggccgagaccattctcgataatattactcagtctacacagtcctttaacgacttcactcgggtggtgggcggagaggacgcaaagcctggccagtttccttggcaggtcgtgctcaacggaaaggtggacgcattttgtggcggatctatcgtgaatgagaagtggattgtgaccgccgctcactgcgtcgagacaggcgtcaagattaccgtggtcgctggagagcacaacattgaggaaacagagcacaccgagcagaagcgcaatgtgattcgcatcatcccacaccacaactacaacgccgctattaataagtacaaccacgacatcgccctgttagaactagatgagcctctggtcctgaatagttacgtcactcctatttgtatcgccgataaagaatacactaacatctttctcaagttcggctctggctatgtatctggatggggacgggtgtttcacaagggacggtccgcactcgtgctacaatacctccgggtgccactggtggatcgggcaacatgcctccggtctaccaagtttactatatacaataacatgttttgcgccggctttcacgagggcggacgggatagttgccagggcgatagtggaggccctcatgtcacagaggtggagggaactagctttctgacaggcattatctcttggggcgaagagtgcgctatgaagggaaagtacggcatctatactaaggtctcgcgttacgtcaattggatcaaggaaaagacaaagctgacaggcggcggagggagcggcggcggaggctctggcggcggcggatctggcggaggcggctctggcggaggcggatctgtcgctccaccacctgcactccccgccgtggctcctccacctgctttgcccgctggatccgtcgctccaccacctgcactccccgccgtggctcctccacctgctttgcccgctgataagacccacacatgtccaccttgcccagcacctgagctgctgggcggacctagcgtgtttctgttcccaccaaagccaaaggacaccctcatgattagtagaaccccagaggtgacttgcgtcgtggtcgatgtcagtcacgaggaccccgaggtgaagtttaattggtatgtagatggcgtcgaggtgcacaacgctaagaccaagccacgcgaagagcagtacgcatctacttaccgggtcgtctctgtgctgactgtgctgcaccaggattggctcaatggcaaagagtacaagtgtaaggtcagtaataaggccctccccgcacctattgaaaagactattagcaaggcaaagggccagcctagggagccccaggtctacaccctgcccccttcccgcgatgagctgactaagaatcaggtctctctcacttgcctcgtgaagggcttttacccatccgacattgccgtggagtgggagtccaacggacagccagagaataactacaagacaactcctcccgtgctcgatagcgacggttcattcttcctgtactccaagctcaccgttgataagtctcggtggcagcagggaaatgtgttctcttgttccgtcatgcacgaggcactgcacaatcactacacccagaagtccctgtccctcagccctggcggcagcggcggatctggaggcggcggatctctcccagagaccggcggataa

SEQ ID NO: 28 - protein, 728 aa, Artificial sequence
  FIX-GS-Fc-L2-P
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGGGSLPETGG

SEQ ID NO: 29 - DNA, 2187 bp, Artificial sequence
  FIX-GS-Fc-L2-P
atgcagcgcgtcaatatgattatggccgagtctccaggcctcattaccatttgtctgctgggctacctgctgtccgcagagtgtaccgtcttcctcgaccacgagaacgcaaataagattctcaataggcctaagcgctacaattccggaaagctcgaagagtttgtccagggaaatctggagcgcgagtgtatggaggaaaagtgtagctttgaagaagctagagaggtgtttgagaatactgagcgcactactgagttttggaagcagtatgtggatggcgatcagtgcgagtccaatccatgcctgaatggcggatcttgcaaggatgatattaattcttacgagtgttggtgcccattcggctttgagggaaagaattgcgaactggatgtcacatgtaacatcaagaacggcagatgcgagcagttttgtaagaacagcgccgataataaggtggtgtgtagttgtactgagggataccgcctcgcagagaatcagaagtcctgtgagcccgctgtgccatttccttgtggccgggtcagcgtcagccagacttctaagctgacacgcgccgagaccgtctttccagatgtggattatgtcaactctactgaggccgagaccattctcgataatattactcagtctacacagtcctttaacgacttcactcgggtggtgggcggagaggacgcaaagcctggccagtttccttggcaggtcgtgctcaacggaaaggtggacgcattttgtggcggatctatcgtgaatgagaagtggattgtgaccgccgctcactgcgtcgagacaggcgtcaagattaccgtggtcgctggagagcacaacattgaggaaacagagcacaccgagcagaagcgcaatgtgattcgcatcatcccacaccacaactacaacgccgctattaataagtacaaccacgacatcgccctgttagaactagatgagcctctggtcctgaatagttacgtcactcctatttgtatcgccgataaagaatacactaacatctttctcaagttcggctctggctatgtatctggatggggacgggtgtttcacaagggacggtccgcactcgtgctacaatacctccgggtgccactggtggatcgggcaacatgcctccggtctaccaagtttactatatacaataacatgttttgcgccggctttcacgagggcggacgggatagttgccagggcgatagtggaggccctcatgtcacagaggtggagggaactagctttctgacaggcattatctcttggggcgaagagtgcgctatgaagggaaagtacggcatctatactaaggtctcgcgttacgtcaattggatcaaggaaaagacaaagctgacaggcggcggagggagcggcggcggaggctctggcggcggcggatctggcggaggcggctctggcggaggcggatccgataagacccacacatgtccaccttgcccagcacctgagctgctgggcggacctagcgtgtttctgttcccaccaaagccaaaggacaccctcatgattagtagaaccccagaggtgacttgcgtcgtggtcgatgtcagtcacgaggaccccgaggtgaagtttaattggtatgtagatggcgtcgaggtgcacaacgctaagaccaagccacgcgaagagcagtacgcatctacttaccgggtcgtctctgtgctgactgtgctgcaccaggattggctcaatggcaaagagtacaagtgtaaggtcagtaataaggccctccccgcacctattgaaaagactattagcaaggcaaagggccagcctagggagccccaggtctacaccctgcccccttcccgcgatgagctgactaagaatcaggtctctctcacttgcctcgtgaagggcttttacccatccgacattgccgtggagtgggagtccaacggacagccagagaataactacaagacaactcctcccgtgctcgatagcgacggttcattcttcctgtactccaagctcaccgttgataagtctcggtggcagcagggaaatgtgttctcttgttccgtcatgcacgaggcactgcacaatcactacacccagaagtccctgtccctcagccctggcggcagcggcggatctggaggcggcggatctctcccagagaccggcggataa

SEQ ID NO: 30 - DNA, 37 bp, Artificial sequence
  F1
tcaggaagatctcgcgaattaattctgtggaatgtgt

SEQ ID NO: 31 - DNA, 36 bp, Artificial sequence
  R1
tatcctagatctgctggcacgacaggtttcccgact

SEQ ID NO: 32 - DNA, 87 bp, Artificial sequence
  F2
ttcgcggatccgtcgctccaccacctgcactccccgccgtggctcctccacctgctttgcccgctgataagacccacacatgtccac

SEQ ID NO: 33 - DNA, 87 bp, Artificial sequence
  R2
taaccggaattcattatccgccggtctctgggagagatccgccgcctccagatccgccgctgccgccagggctgagggacagggact

SEQ ID NO: 34 - DNA, 48 bp, Artificial sequence
  F3
tggctcctccacctgctttgcccgctgtcgctccaccacctgcactcc

SEQ ID NO: 35 - DNA, 57 bp, Artificial sequence
  F4
tagatctgtcgctccaccacctgcactccccgccgtggctcctccacctgctttgcc

SEQ ID NO: 36 - DNA, 31 bp, Artificial sequence
  R3
tgaattcttatcatccgccggtctctgggag

SEQ ID NO: 37 - DNA, 47 bp, Artificial sequence
  F5
ccctgtcagactgcctggacccgctgtcgctccaccacctgcactcc

SEQ ID NO: 38 - DNA, 54 bp, Artificial sequence
  F6
tagatctgtcgctccaccacctgcactccccgcccctgtcagactgcctggacc

SEQ ID NO: 39 - DNA, 27 bp, Artificial sequence
  F7
atataagcttccgccaccatgcagcgc

SEQ ID NO: 40 - DNA, 34 bp, Artificial sequence
  R4
atatggatccagcgggcaaagcaggtggaggagc

SEQ ID NO: 41 - DNA, 37 bp, Artificial sequence
  F8
gatttggatccgataagacccacacatgtccaccttg

SEQ ID NO: 42 - DNA, 87 bp, Artificial sequence; identical to SEQ ID NO: 33
  R5

(19) United States
(12) Patent Application Publication
(10) Pub. No.: US 2022/0323590 A1
(43) Pub. Date: Oct. 13, 2022

(54) IMPROVED FIX FUSION PROTEIN AND CONJUGATE AND USE THEREOF

(71) Applicant: JIANGSU GENSCIENCES INC., Nantong (CN)

(72) Inventors: Hongsheng SU, Beijing (CN); Xian CHEN, Beijing (CN); Weichuan MO, Beijing (CN); Luyan ZHU, Beijing (CN); Haixia YAN, Beijing (CN); Zijia REN, Beijing (CN); Yali WANG, Beijing (CN)

(73) Assignee: JIANGSU GENSCIENCES INC., Nantong (CN)

(21) Appl. No.: 17/597,149

(22) PCT Filed: Jun. 24, 2020
(86) PCT No.: PCT/CN2020/097911

§ 371 Date: Dec. 28, 2021

(30) Foreign Application Priority Data
Jul. 2, 2019 (CN) 201910591280.0

(51) Int. Cl. A61K 47/60; A61K 38/36; A61K 38/48; A61K 47/68; A61P 7/04; C12N 9/64

(52) U.S. Cl. CPC A61K 47/60; A61K 38/36; A61K 38/48; A61K 47/68; A61P 7/04; C12N 9/644; C12Y 304/21022; C07K 2319/30; C07K 2319/31

(57) ABSTRACT

Provided are a blood coagulation factor IX fusion protein with a prolonged circulation half-life, a conjugate thereof, a pharmaceutical composition containing same and the use of same in treating hemorrhagic diseases (such as hemophilia B).

### CROSS REFERENCE TO RELATED APPLICATIONS

This application claims the priority of Chinese Patent Application No. 201910591280.0, filed with the China National Intellectual Property Administration on Jul. 2, 2019, and the disclosure of which is hereby incorporated by reference in its entirety.

### FIELD

The present disclosure relates to a biologically active fusion protein with extended half-life in vivo and a conjugate thereof with a polymer, and specifically relates to human coagulation factor IX with extended half-life in vivo, a pharmaceutical composition containing the same, and the use of the same in the treatment of hemorrhagic diseases (such as hemophilia B).

### BACKGROUND

Hemophilia, a severe coagulation dysfunction caused by the deficiency of certain coagulation factors in the blood, is a set of hereditary hemorrhagic diseases, including hemophilia A (factor VIII, AHG deficiency) and hemophilia B (factor IX, PTC deficiency).

Hemophilia B is a serious coagulation dysfunction disease caused by the deficiency of human coagulation factor IX (hFIX). The gene encoding hFIX (hFIX) is located at the far end of the long arm of human X chromosome, the Xq27.1 region, with a full length of 34 kb, containing 8 exons and 7 introns. The cDNA of hFIX has a full length of 2802 bp, of which the coding region is 1383 bp (Genbank accession number: NM_000133). The severe patients regularly have an activity of factor IX lower than 1% of normal, who often suffered from spontaneous bleeding which leads to muscle hematoma or joint deformity.

Recombinant formulations for the prevention and treatment of hemophilia are increasingly recognized by the medical system and patients for their safety. For hemophilia B, infusion of factor IX preparation (which is usually recombinant factor IX protein expressed in vitro at present) is the only effective treatment method to supplement the coagulation factor IX level in the patient's body. However, since peptide drugs generally have characteristics such as short half-life, poor physical and chemical stability, and easy degradation by various proteases in the body, these drugs usually require multiple injections within a day, resulting in frequent medication, which brings a greater physical, mental and economic burden to patients, limiting the patient's medication compliance. Therefore, there is an urgent need for new drugs in this field to extend the circulation half-life in plasma and increase the systemic drug exposure.

### SUMMARY

After years of research, the inventors invent a fusion protein of coagulation factor IX and Fc, which can be further modified by a group to be a conjugate, including PEG, and has a significantly extended circulation half-life compared with FIX polypeptides.

Specifically, the first aspect of the present disclosure provides a fusion protein of coagulation factor IX (FIX), comprising a coagulation factor IX active moiety and a fusion partner (FP) capable of extending the half-life of the fusion protein, wherein the coagulation factor IX active moiety and the fusion partner are linked directly or through a first linker L1; preferably, the first linker L1 is a peptide containing 1, 2, 3, 4, 5, 6, 7, 8, 9, 10, or more amino acids; more preferably, the first linker L1 comprises a flexible peptide and/or a rigid unit, and the flexible peptide is a peptide containing glycine (Gly, G), serine (Ser, S), alanine (Ala, A) and/or threonine (Thr, T), for example, the flexible peptide is (GS)ₘ(GGS)ₙ(GGGS)ₒ(GGGGS)ₚ, where m, n, o and p are independently selected from integers from 0 to 50, such as 0, 1, 2, 3, 4, 5, 6, 7, 8, 9, 10, 11, 12, 13, 14, 15, 16, 17, 18, 19 or 20, preferably m, n, o and p are not all O.

In one embodiment of the present disclosure, the coagulation factor IX active moiety is derived from human, such as full-length or truncated human coagulation factor IX; the full-length or truncated human coagulation factor IX may contain one or more amino acid mutations, provided that it still retains the FIX activity, for example, the coagulation factor IX active moiety comprises the amino acid sequence shown in SEQ ID NO: 1, or has at least 80%, 85%, 88%, 89%, 90%, 91%, 92%, 93%, 94%, 95%, 96%, 97%, 98%, 99% or higher identity to the amino acid sequence shown in SEQ ID NO: 1.

In another embodiment, the rigid unit is a peptide containing 1, 2, 3, 4, 5, 6, 7, 8, 9, 10 or more proline (Pro, P), preferably, the rigid unit comprises an amino acid sequence selected from the group consisting of:

(SEQ ID NO: 3)
VAPPPALPAPVRLPGPA,
(SEQ ID NO: 4)
VAPPPALPAVAPPPALPA,
(SEQ ID NO: 5)
VAPPPALPAVAPPPALPAVAPPPALPAPVRLPGPA,
(SEQ ID NO: 6)
VAPPPALPAPVRLPGPAVAPPPALPAVAPPPALPA,
and
(SEQ ID NO: 7)
VAPPPALPAVAPPPALPAGSVAPPPALPAVAPPPALPA.

Preferably, the first linker L1 is selected from the group consisting of:

(SEQ ID NO: 8)
GGGGSGGGGSGGGGSGGGGSGGGGSVAPPPALPAPVRLPGPA,
(SEQ ID NO: 9)
GGGGSGGGGSGGGGSGGGGSGGGGSVAPPPALPAVAPPPALPA,
(SEQ ID NO: 10)
GGGGSGGGGSGGGGSGGGGSGGGGSVAPPPALPAVAPPPALPAVAPPPAL
PAPVRLPGPA,
(SEQ ID NO: 11)
GGGGSGGGGSGGGGSGGGGSGGGGSVAPPPALPAPVRLPGPAVAPPPALP
AVAPPPALPA,
and
(SEQ ID NO: 12)
GGGGSGGGGSGGGGSGGGGSGGGGSVAPPPALPAVAPPPALPAGSVAPPP
ALPAVAPPPALPA.

In yet another embodiment, the fusion partner is a full-length, truncated or variant form of immunoglobulin Fc fragment, albumin, transferrin or XTEN; the fusion partner is, for example, derived from human, for example, the Fc fragment is derived from a human immunoglobulin Fc fragment; preferably, the Fc fragment is composed of one to four domains selected from the group consisting of CH1 domain, CH2 domain, CH3 domain and CH4 domain; preferably, the Fc fragment is derived from the Fc fragment of IgG, IgA, IgD, IgE, or IgM, more preferably IgG Fc fragment; more preferably, the Fc fragment is derived from the Fc fragment of IgG1, IgG2, IgG3, or IgG4, and more preferably, the IgG Fc fragment has reduced ADCC effect and/or CDC effect and/or enhanced binding affinity to FcRn.

In yet another embodiment, wherein the fusion partner (FP) is also linked directly or via a second linker L2 to a peptide P containing a Sortase recognition site, and the Sortase is, for example, Sortase A or Sortase B; preferably, the P contains the core recognition site LPXTG of Sortase A, wherein X is any amino acid, and the sequence of P is, such as LPETG, LPETGG or LPETGGG; more preferably, the amino acid sequence of P further contains an affinity tag linked to the Sortase recognition site, and the amino acid sequence of P is, for example, LPETGGHHHHHH or LPETGGWSHPQFEK.

In yet another embodiment, the second linker L2 is a peptide containing 1, 2, 3, 4, 5, 6, 7, 8, 9, 10 or more amino acids; preferably, the second linker L2 is a flexible peptide fragment containing glycine (Gly, G), serine (Ser, S), alanine (Ala, A) and/or threonine (Thr, T), such as (GS)_{w}(GGS)ₓ(GGGS)_{y}(GGGGS)_{z}, where w, x, y and z are independently selected from integers from 0 to 50, such as 0, 1, 2, 3, 4, 5, 6, 7, 8, 9, 10, 11, 12, 13, 14, 15, 16, 17, 18, 19 or 20, preferably w, x, y and z are not all 0; preferably, the second linker L2 is selected from the group consisting of GGGGS (SEQ ID NO: 14), GGGGSGGGGS (SEQ ID NO: 15) and GSGGSGGGGS (SEQ ID NO: 16).

In another embodiment, the fusion protein has a structure of FIX-L1-FP-L2-P, wherein FIX, L1, L2, FP, and P have the same definitions as in the above embodiments, and wherein either or both of L1 and L2 may not be present.

In yet another embodiment, the fusion protein comprises the sequence shown in SEQ ID NO: 18, 20, 22, 24, 26 or 28, or encoded by the nucleic acid sequence shown in SEQ ID NO: 19, 21, 23, 25, 27, or 29.

Further, the present disclosure also provides a nucleic acid molecule encoding the fusion protein of any one of the above embodiments and an expression vector comprising the nucleic acid molecule.

The second aspect of the present disclosure provides a conjugate, wherein the conjugate is formed by linking a hydrophilic polymer to the end of the fusion protein according to any embodiment of the first aspect. In the conjugate, one, two or more hydrophilic polymers are attached to the end of the fusion protein according to any embodiment of the first aspect, preferably, the hydrophilic polymer is attached to the Sortase recognition site via a transpeptidation reaction; in particular, the fusion protein may be in a form of monomer or dimer.

In one embodiment, the one, two or more hydrophilic polymers are each independently selected from polysaccharide and polyalkylene glycol, such as polypropylene glycol and polyethylene glycol; the polyalkylene glycol may be end-capped, for example, capped with alkoxy group such as methoxy group; and/or the polyalkylene glycol is linear or branched, for example, the polyalkylene glycol is branched, such as branched polyethylene glycol, especially branched polyethylene glycol capped with methoxy group; the molecular weight of the polyalkylene glycol may be ≥1, ≥10, ≥20, ≥30, ≥40, ≥50, ≥60, ≥70, ≥80, ≥90, ≥100, ≥110, ≥120, ≥130, ≥140, ≥150, or ≥160 kDa, for example, 5, 10, 20, 30, 40, 50, 60, 70, 80, 90, or 100 kDa, or a value between any two of the values; and preferably the molecular weight of the hydrophilic polymer is 20 kDa or 40 kDa.

The third aspect of the present disclosure provides a method for producing the conjugate of the second aspect, comprising contacting the fusion protein of the first aspect of the present disclosure with Sortase (such as Sortase A or Sortase B) and the hydrophilic polymer, wherein one end of the hydrophilic polymer has an amino group that can be amidated by Sortase.

In one embodiment, the N-terminus of the hydrophilic polymer contains poly-Gly, such as GGGAA.

The fourth aspect of the present disclosure provides a pharmaceutical composition comprising an effective amount of the fusion protein described in the first aspect and/or the conjugate described in the second aspect, and optionally a pharmaceutically acceptable carrier.

In one embodiment, the pharmaceutical composition is used for the prevention and/or treatment of a hemorrhagic disease, wherein the hemorrhagic disease is preferably selected from hemorrhagic diseases in patients with congenital or acquired deficiency of FIX, and spontaneous or surgical bleeding in patients with hemophilia B.

In yet another embodiment, the pharmaceutical composition is in a form of liquid preparation or lyophilized preparation.

The fifth aspect of the present disclosure provides use of the fusion protein, conjugate or pharmaceutical composition of the aforementioned aspects for the manufacture of a medicament for the prevention and/or treatment of a hemorrhagic disease, wherein the hemorrhagic disease is preferably selected from hemorrhagic diseases in patients with congenital or acquired deficiency of FIX, and spontaneous or surgical bleeding in patients with hemophilia B.

The sixth aspect of the present disclosure provides a method for preventing and/or treating a hemorrhagic disease, comprising administering the fusion protein, conjugate or pharmaceutical composition of the aforementioned aspects to a subject in need thereof. The hemorrhagic disease is preferably selected from hemorrhagic diseases in patients with congenital or acquired deficiency of FIX, and spontaneous or surgical bleeding in patients with hemophilia B.

The seventh aspect of the present disclosure provides a kit comprising the fusion protein, conjugate or pharmaceutical composition of the aforementioned aspects, and optionally instructions for use.

The eighth and ninth aspects of the present disclosure respectively provide a nucleic acid molecule encoding the fusion protein of the first aspect and an expression vector comprising the nucleic acid molecule.

### BRIEF DESCRIPTION OF DRAWINGS

FIG. 1A shows the structure of the FIX/PACE (Furin) bicistronic expression vector.

FIG. 1B shows the plasmid map of pcDNA3.1-Hyg-VKGC.

FIG. 2 shows the detection results of SDS-PAGE (A) and SEC-HPLC (B) of the purified FIX-C1-Fc-L2-P fusion protein.

FIG. 3 shows the detection results of the in vitro activity of each FIX-L1-Fc-L2-P fusion protein and FIX-C1-Fc-L2-P conjugate.

FIG. 4 shows the SEC-HPLC detection result of the 40 kDa PEG conjugate after the conjugation reaction.

FIG. 5 shows the SEC-HPLC detection results of the conjugate after purification and recovery.

FIG. 6 shows the PK detection results of the conjugate FIX-C1-Fc-L2-PEG in hemophilia B model mice, and the table below shows the T1/2 (elimination phase half-life), Cmax, AUC0-inf (area under the curve), MRT (mean residence time) and CL (clearance rate).

### DETAILED DESCRIPTION

In order to make the objectives, technical solutions, and advantages of the present disclosure clearer, the following further describes the present disclosure in detail with reference to the drawings and embodiments. Obviously, the embodiments to be described merely are a part rather than all of the embodiments of present disclosure. Based on the embodiments of the present disclosure, all other embodiments obtained by those of ordinary skill in the art without creative work shall fall within the protection scope of the present disclosure.

### Definitions

Coagulation factor IX, also called factor IX, FIX, is a vitamin K-dependent coagulation factor with structural similarities to factor VII, prothrombin, factor X, and protein C.

In the present disclosure, the term “coagulation factor IX active moiety” refers to the moiety of the fusion protein that exhibits coagulation factor IX activity. The term “coagulation factor IX” can refer to its natural wild-type protein (the wild-type amino acid sequence is shown in SEQ ID NO:1, and the reference wild-type nucleic acid coding sequence can be found in

Genbank accession number: NM_000133, specifically as shown in SEQ ID NO: 2), and also encompasses its variant forms, for example, a variant protein generated by one or more (such as 2, 3, 4 or 5) amino acid substitutions, deletions or insertions, while retaining the activity of coagulation factor IX.

(SEQ ID NO: 1)
MQRVNMIMAESPGLITICLLGYLLSAECTVFLDHENANKILNRPKRYNSG
KLEEFVQGNLERECMEEKCSFEEAREVFENTERTTEFWKQYVDGDQCESN
PCLNGGSCKDDINSYECWCPFGFEGKNCELDVTCNIKNGRCEQFCKNSAD
NKVVCSCTEGYRLAENQKSCEPAVPFPCGRVSVSQTSKLTRAETVFPDVD
YVNSTEAETILDNITQSTQSFNDFTRVVGGEDAKPGQFPWQVVLNGKVDA
FCGGSIVNEKWIVTAAHCVETGVKITVVAGEHNIEETEHTEQKRNVIRII
PHHNYNAAINKYNHDIALLELDEPLVLNSYVTPICIADKEYTNIFLKFGS
GYVSGWGRVFHKGRSALVLQYLRVPLVDRATCLRSTKFTIYNNMFCAGFH
EGGRDSCQGDSGGPHVTEVEGTSFLTGIISWGEECAMKGKYGIYTKVSRY
VNWIKEKTKLT.
(SEQ ID NO: 2)
ATGCAGCGCGTGAACATGATCATGGCAGAATCACCAGGCCTCATCACCAT
CTGCCTTTTAGGATATCTACTCAGTGCTGAATGTACAGTTTTTCTTGATC
ATGAAAACGCCAACAAAATTCTGAATCGGCCAAAGAGGTATAATTCAGGT
AAATTGGAAGAGTTTGTTCAAGGGAACCTTGAGAGAGAATGTATGGAAGA
AAAGTGTAGTTTTGAAGAAGCACGAGAAGTTTTTGAAAACACTGAAAGAA
CAACTGAATTTTGGAAGCAGTATGTTGATGGAGATCAGTGTGAGTCCAAT
CCATGTTTAAATGGCGGCAGTTGCAAGGATGACATTAATTCCTATGAATG
TTGGTGTCCCTTTGGATTTGAAGGAAAGAACTGTGAATTAGATGTAACAT
GTAACATTAAGAATGGCAGATGCGAGCAGTTTTGTAAAAATAGTGCTGAT
AACAAGGTGGTTTGCTCCTGTACTGAGGGATATCGACTTGCAGAAAACCA
GAAGTCCTGTGAACCAGCAGTGCCATTTCCATGTGGAAGAGTTTCTGTTT
CACAAACTTCTAAGCTCACCCGTGCTGAGACTGTTTTTCCTGATGTGGAC
TATGTAAATTCTACTGAAGCTGAAACCATTTTGGATAACATCACTCAAAG
CACCCAATCATTTAATGACTTCACTCGGGTTGTTGGTGGAGAAGATGCCA
AACCAGGTCAATTCCCTTGGCAGGTTGTTTTGAATGGTAAAGTTGATGCA
TTCTGTGGAGGCTCTATCGTTAATGAAAAATGGATTGTAACTGCTGCCCA
CTGTGTTGAAACTGGTGTTAAAATTACAGTTGTCGCAGGTGAACATAATA
TTGAGGAGACAGAACATACAGAGCAAAAGCGAAATGTGATTCGAATTATT
CCTCACCACAACTACAATGCAGCTATTAATAAGTACAACCATGACATTGC
CCTTCTGGAACTGGACGAACCCTTAGTGCTAAACAGCTACGTTACACCTA
TTTGCATTGCTGACAAGGAATACACGAACATCTTCCTCAAATTTGGATCT
GGCTATGTAAGTGGCTGGGGAAGAGTCTTCCACAAAGGGAGATCAGCTTT
AGTTCTTCAGTACCTTAGAGTTCCACTTGTTGACCGAGCCACATGTCTTC
GATCTACAAAGTTCACCATCTATAACAACATGTTCTGTGCTGGCTTCCAT
GAAGGAGGTAGAGATTCATGTCAAGGAGATAGTGGGGGACCCCATGTTAC
TGAAGTGGAAGGGACCAGTTTCTTAACTGGAATTATTAGCTGGGGTGAAG
AGTGTGCAATGAAAGGCAAATATGGAATATATACCAAGGTATCCCGGTAT
GTCAACTGGATTAAGGAAAAAACAAAGCTCACTTAA.

In one embodiment, the variant protein is at least 90% identical to the sequence of SEQ ID NO: 1. In another embodiment, the variant protein is at least 95%, such as 96%, 97%, 98%, or 99% identical to the sequence of SEQ ID NO: 1. As used herein, unless otherwise specified, any specific position mentioned refers to the corresponding position in SEQ ID NO: 1.

The present disclosure is not limited to the sequences described herein. FIX variants are, for example, disclosed in U.S. Pat. No. 5,521,070 (wherein the tyrosine in the first position is substituted by alanine) and WO2007/135182 (wherein one or more natural amino acid residues in FIX are substituted by cysteine residues). The references are incorporated herein by reference in their entirety. Therefore, FIX variants are well known in the art, and the present disclosure encompasses those variant forms known or will be developed or discovered in the future.

### Fusion Protein

The term “fusion partner”, FP, refers to a polypeptide that is fused with a target polypeptide (a polypeptide whose circulation half-life is desired to be extended). The fusion partner can affect the function properties of the fusion protein through a variety of different mechanisms, such as extending the half-life of the target polypeptide in vivo.

In one embodiment, the fusion partner delays the clearance of FIX in vivo by interacting with the neonatal Fc receptor (FcRn). In another embodiment, the fusion partner is an immunoglobulin Fc fragment, albumin, transferrin, XTEN, or a portion thereof.

The Fc fragment of immunoglobulin is safe to be used as a pharmaceutical carrier because it is a biodegradable polypeptide that can be metabolized in the body. In addition, compared with the entire immunoglobulin molecule, the Fc region of immunoglobulin has a relatively low molecular weight, which is beneficial to the preparation, purification and production of the conjugate. Since the immunoglobulin Fc fragment does not contain Fab fragment (its amino acid sequence varies according to the antibody subclass and is therefore highly heterogeneous), it is expected that the immunoglobulin Fc region can greatly increase the homogeneity of the substance and have low antigenicity.

The term “Fc region/fragment of immunoglobulin” refers to a protein containing heavy chain constant region 2 (CH2) and heavy chain constant region 3 (CH3) of immunoglobulin, but not variable regions of heavy chain and light chain of an immunoglobulin. Furthermore, the Fc fragment used in the present disclosure may further contain the hinge region in the heavy chain constant region, and/or part or all of the heavy chain constant region 1 (CH1) and/or the light chain constant region 1 (CL1) without variable regions of heavy chain and light chain, as long as it has a physiological function that is basically similar to or better than that of natural form.

In some embodiments, the immunoglobulin Fc fragment of the present disclosure may comprise 1) CH1 domain, CH2 domain, CH3 domain and CH4 domain; 2) CH1 domain and CH2 domain; 3) CH1 domain and CH3 domain; 4) CH2 domain and CH3 domain; 5) the combination of one or more domains with (part or all of) the immunoglobulin hinge region; or 6) a dimer of any domains of heavy chain constant region and light chain constant region.

In addition, the immunoglobulin Fc fragment may be an Fc region derived from IgG, IgA, IgD, IgE, and IgM, or prepared by a combination or hybrid thereof. Preferably, it is derived from IgG or IgM (because they are two of the most abundant proteins in human blood). In one embodiment, considering that IgG antibodies have a longer half-life, the IgG Fc domain is preferred.

The Fc fragment can also be modified to improve other functions, for example, to change its ability to induce immune responses by mutations, such as the ability to bind to complement and/or to some Fc receptors, because this is unnecessary for the use of Fc to extend the half-life. It is known that the mutations at positions 234, 235, and 237 in the Fc region of IgG usually lead to reduced binding to FcγRI receptors, and may also lead to reduced binding to FcγRIIa and FcγRIII receptors, but these mutations do not change its binding to FcRn receptors, and can therefore still promote a long circulation half-life through the endocytic recirculation pathway. Preferably, the modified IgG Fc fragment of the fusion protein of the present disclosure contains one or more of the following mutations, mutations L234A, L235E and G237A lead to decreased affinity for certain Fc receptors, and A330S and P331S lead to decreased C1q-mediated complement binding.

In one embodiment, the coagulation factor IX fusion protein is further linked to a fragment containing a Sortase recognition site, i.e., the “peptide P” described in the present disclosure.

The term “Sortase”, specifically including Sortase A and Sortase B, was first discovered to have the function of anchoring bacterial surface proteins on the cell wall. The “sorting signal” of the surface proteins was identified to consist of three key parts: the LPXTG sequence, the hydrophobic sequence and a tail of positively charged residues in most cases. The LPXTG sequence is very conservative and can be recognized by Sortase, then the cysteine (Cys) of the Sortase acts as a nucleophilic group to attack the peptide bond between the threonine and glycine of the C-terminal motif LPXTG of the substrate (such as surface protein), causing the peptide bond to cleave (acylation), which in turn produces an acylase intermediate, and finally the peptide is covalently linked to the cell wall or fimbriae subunit to function (deacylation)

In one embodiment of the present disclosure, Sortase A is utilized for site-directed coupling of PEG. In a specific embodiment, the Sortase A core recognition sites LPETG, LPETGG, and LPETGGG are used. In another specific embodiment, different affinity tags can be added to the recognition site, such as LPETGGHHHHHH, LPETGGWSHPQFEK, etc.

In another embodiment, the coagulation factor IX fusion protein of the present disclosure is produced by recombinant methods, comprising, for example, expressing the protein in a suitable prokaryotic or eukaryotic host cell, and isolating the coagulation factor IX fusion protein of the present disclosure by conventional techniques. For example, the nucleotide sequence encoding the peptide can be synthesized by chemical synthesis method first, and then the sequence can be cloned into a suitable expression vector for expression under the control of a suitable promoter. Alternatively, mutagenesis methods such as PCR mutagenesis can be employed to obtain the nucleotide sequence encoding coagulation factor IX from wild-type coagulation factor IX, and then the sequence and the sequence of other elements for constructing the fusion protein can be cloned to a suitable expression vector for expression under the control of a suitable promoter. These technologies are completely within the abilities of those of ordinary skill in the art, and there are numerous teachings in the prior art.

Suitable eukaryotic host cells comprise mammalian cells, such as CHO, COS, HEK 293, BHK, SK-Hep and HepG2. The cells are preferably growing under conditions suitable for expressing the coagulation factor IX fusion protein of the present disclosure. As for the reagents and conditions used to produce or isolate the coagulation factor IX fusion protein of the present disclosure, there are no special restrictions, and any system known in the art or commercially available can be applied. In a preferred embodiment, the coagulation factor IX fusion protein is obtained by methods described in the art.

There are a variety of expression vectors that can be used to prepare the coagulation factor IX fusion protein, which can be selected from eukaryotic and prokaryotic expression vectors. Prokaryotic expression vectors may include, for example, plasmids such as pRSET, pET, and pBAD, in which promoters that can be used include, for example, lac, trc, trp, recA, or araBAD. Eukaryotic expression vectors include: (i) vectors used for expression in yeast, such as pAO, pPIC, pYES, and pMET, in which promoters such as AOX1, GAP, GAL1, AUG1, etc. can be used; (ii) vectors used for expression in insect cells, such as pMT, pAc[delta], p1B, pMIB, pBAC, etc., in which promoters such as PH, p10, MT, Ac5, OplE2, gp64, polh, etc. can be used; and (iii) vectors used for expression in mammalian cells, such as pSVL, pCMV, pRc/RSV, pcDNA3, pBPV, etc., and vectors derived from viral systems such as vaccinia virus, adeno-associated virus, herpes virus, retrovirus, and the like, in which promoters such as CMV, SV40, EF-1, UbC, RSV, ADV, BPV and β-actin can be used. In a preferred embodiment, the coagulation factor IX fusion protein is expressed in a prokaryotic or eukaryotic cell system, and codon-optimized sequences are used.

In a preferred embodiment, the sequence for expressing the coagulation factor IX fusion protein contains a propeptide and/or a signal peptide to facilitate the secretion of the coagulation factor IX fusion protein from the cell to the outside of the cell for separation and purification; in order to improve the cleavage efficiency of the propeptide, the yield of FIX and the correct processing, the expression vector was optimized; in a specific embodiment, a FIX/PACE (Furin) bicistronic expression vector was constructed.

In another preferred embodiment, the sequence expressing the coagulation factor IX fusion protein does not contain a propeptide and/or a signal peptide, and instead of being secreted outside the cell, the coagulation factor IX fusion protein is obtained by lysing the cell for separation and purification.

In an embodiment, in the fusion protein of the present disclosure, the coagulation factor IX protein and the Fc fragment are directly linked. In another embodiment, the coagulation factor

IX protein and the Fc fragment are linked through a first linker L1. In another embodiment, the Fc fragment and the fragment containing the Sortase recognition sequence are directly linked. In another embodiment, the Fc fragment and the fragment containing the Sortase recognition sequence are linked through a second linker L2.

In preferred embodiments, the first linker L1 is a peptide containing 1, 2, 3, 4, 5, 6, 7, 8, 9, 10, or more amino acids; more preferably, the first linker L1 comprises a flexible peptide and/or a rigid unit, and the flexible peptide is a peptide containing glycine (Gly, G), serine (Ser, S), alanine (Ala, A) and/or threonine (Thr, T), for example, the flexible peptide is (GS)ₘ(GGS)ₙ(GGGS)ₒ(GGGGS)ₚ, where m, n, o and p are independently selected from integers from 0 to 50, such as 0, 1, 2, 3, 4, 5, 6, 7, 8, 9, 10, 11, 12, 13, 14, 15, 16, 17, 18, 19 or 20, preferably m, n, o and p are not all 0.

The rigid unit is a peptide containing one or more prolines (Pro, P). Proline has a special cyclic structure, which strengthens the restriction on the structure of the peptide chain. The inventors unexpectedly discovered that the inclusion of a rigid unit in the linker can greatly improve the activity of the fusion protein. Preferably, the rigid unit comprises an amino acid sequence selected from the group consisting of:

(SEQ ID NO: 3)
VAPPPALPAPVRLPGPA,
(SEQ ID NO: 4)
VAPPPALPAVAPPPALPA,
(SEQ ID NO: 5)
VAPPPALPAVAPPPALPAVAPPPALPAPVRLPGPA,
(SEQ ID NO: 6)
VAPPPALPAPVRLPGPAVAPPPALPAVAPPPALPA,
and
(SEQ ID NO: 7)
VAPPPALPAVAPPPALPAGSVAPPPALPAVAPPPALPA.

In another preferred embodiment, the sequence of the first linker L1 is selected from the group consisting of:

(SEQ ID NO: 8)
GGGGSGGGGSGGGGSGGGGSGGGGSVAPPPALPAPVRLPGPA,
(SEQ ID NO: 9)
GGGGSGGGGSGGGGSGGGGSGGGGSVAPPPALPAVAPPPALPA,
(SEQ ID NO: 10)
GGGGSGGGGSGGGGSGGGGSGGGGSVAPPPALPAVAPPPALPAVAPPPA
LPAPVRLPGPA,
(SEQ ID NO: 11)
GGGGSGGGGSGGGGSGGGGSGGGGSVAPPPALPAPVRLPGPAVAPPPAL
PAVAPPPALPA,
and
(SEQ ID NO: 12)
GGGGSGGGGSGGGGSGGGGSGGGGSVAPPPALPAVAPPPALPAGSVAPP
PALPAVAPPPALPA.

The second linker L2 is a peptide containing 1, 2, 3, 4, 5, 6, 7, 8, 9, 10 or more amino acids, and is mainly used to separate Fc and the Sortase recognition site to facilitate the recognition and action of Sortase; more preferably, the second linker L2 is a flexible peptide containing glycine (Gly, G), serine (Ser, S), alanine (Ala, A) and/or threonine (Thr, T), such as (GS)_{w}(GGS)ₓ(GGGS)_{y}(GGGGS)_{z}, where w, x, y and z are independently selected from integers from 0 to 50, such as 0, 1, 2, 3, 4, 5, 6, 7, 8, 9, 10, 11, 12, 13, 14, 15, 16, 17, 18, 19 or 20, preferably w, x, y and z are not all 0.

In a preferred embodiment, the sequence of the second linker L2 is selected from the group consisting of GGGGS (SEQ ID NO: 14), GGGGSGGGGS (SEQ ID NO: 15) and GSGGSGGGGS (SEQ ID NO: 16).

Coagulation factor IX fusion protein conjugate or conjugate of coagulation factor IX fusion protein

The term “conjugate” refers to a product formed by a polypeptide or a polypeptide variant covalently or non-covalently linked to the hydrophilic polymer modification group described herein, wherein the hydrophilic polymer and the polypeptide can be linked at any position, for example, the appropriate position at the N-terminal, C-terminal or middle part of the polypeptide.

Generally, the term “polymer” used herein has the meaning commonly understood by those of ordinary skill in the art, and refers to both the polymer itself and its terminal-modified derivatives, unless explicitly stated otherwise.

In addition, for polymer such as polyethylene glycol, there are many ways to determine their molecular weight. Since polymers are composed of molecules with different degrees of polymerization within a certain distribution range, the molecular weights of the polymers are generally represented by their average molecular weight, specifically, number-average molecular weight or weight-average molecular weight. The number-average molecular weight refers to the sum of the product of the number fraction of each molecule with different molecular weight and its corresponding molecular weight; and the weight-average molecular weight refers to the sum of the product of the weight fraction of each different molecule and its corresponding molecular weight. Although the number-average molecular weight and weight-average molecular weight may have some deviations when the degree of polymerization of the polymers differs greatly, for polymers with a narrow distribution range, the two tend to be equal. For polymers such as polyethylene glycol mentioned herein, when referring to its molecular weight, it can be either weight-average molecular weight or number-average molecular weight.

The coagulation factor IX fusion protein of the present disclosure can be conjugated with one or more hydrophilic polymers to form a coagulation factor IX fusion protein conjugate. The polymer is preferably physiologically acceptable, which includes those soluble in an aqueous solution or suspension and without negative effects such as side effects on mammals after administration of the coagulation factor IX fusion protein conjugate in a pharmaceutically effective amount. The polymers that can be used in the present disclosure are not particularly limited. The polymers generally preferably have 2 to about 3000 repeating units. The polymer group can be selected from natural or synthetic polymers, examples of which include, but are not limited to, for example, polysaccharides, polyalkylene glycols such as polyethylene glycol (PEG), polypropylene glycol (PPG), polyethylene oxide (PEO), copolymer of ethylene glycol and propylene glycol, polyvinyl alcohol and any combination thereof. In a preferred embodiment, the coagulation factor IX fusion protein conjugate of the present disclosure is conjugated with one or more PEG groups for modification.

In the present disclosure, the polymer is not limited to a particular structure, it can be linear (such as alkoxy PEG or bifunctional PEG), branched or multi-armed (such as bifurcated PEG or PEG linked to polyol core), dendritic or may have degradable linkages. In addition, the internal structure of the polymer can be organized in any number of different patterns, which can be selected from homopolymers, alternating copolymers, random copolymers, block copolymers, alternating terpolymers, random terpolymers, block terpolymers, and the like. The polymer may also include poly (alkylene oxide) polymers, polymaleic acid, poly (D, L-alanine), and the like.

In some embodiments, the polymer is polyethylene glycol (PEG) or derivatives thereof such as methoxy polyethylene glycol (mPEG). Herein, unless specifically indicated, the polyethylene glycols (PEG) include those either with hydroxyl groups as the terminal group or with other groups as the terminal group. The other groups include, but are not limited to, alkoxy, cycloalkoxy, cycloalkyloxy, alkenyl, aryloxy or aralkyloxy. These PEG molecular types are known in the prior art and are routinely used in polypeptide modification. The PEG side chain can be linear, branched, bifurcated or composed of multiple arms. Different polyethylene glycols can have different polymer chain lengths and polymer structures.

In the present disclosure, there is no particular limitation on the molecular weight of PEG, and its molecular weight can range from 0.1 to 200 kDa, such as 1 to 150 kDa, 2 to 100 kDa, 3 to 80 kDa, 4 to 50 kDa, or 5 to 40 kDa. Other useful PEG include, for example, those disclosed in WO 03/040211, U.S. Pat. Nos. 6,566,506, 6,864,350, and 6,455,639. In particular, the PEG has a general formula HO—CH₂CH₂O—(CH₂CH₂O)ₙ—CH₂CH₂—OH, wherein the range of n is about 5 to 4000. As mentioned above, the PEG of the present disclosure includes PEG with other terminal groups, such as methoxy PEG, branched PEG, bifurcated PEG, and the like. Suitable branched PEGs can be prepared as described in U.S. Pat. No. 5,932,462, the entire disclosure of which is incorporated herein by reference. The bifurcated PEG refers to a PEG that has a branch near one end of the polymer chain, and the main chain of the bifurcated PEG can be linear or branched.

It is known to those skilled in the art that in a bioactive molecule conjugated with a polymer group, as the molecular weight of the polymer group increases, the biological activity of the conjugate usually gradually decreases. It is also known to those skilled in the art that as the molecular weight of the polymer group increases, the biological half-life and/or plasma half-life of the conjugate increases accordingly.

In order to provide a stable therapeutic effect over a long period, and to reduce the frequency of administration to improve patient compliance, it is desirable to extend the biological half-life of the coagulation factor IX fusion protein conjugate as much as possible while retaining significant activity of the coagulation factor IX receptor agonist. Therefore, the present disclosure provides the coagulation factor IX fusion protein conjugate with extended biological half-life and significant activity of the coagulation factor IX receptor agonist.

In a specific embodiment, in the coagulation factor IX fusion protein conjugate of the present disclosure, the molecular weight of the one or more polymer groups (such as PEG) is ≥1, ≥10, ≥20, ≥30, ≥40, ≥50, ≥60, ≥70, ≥80, ≥90, ≥100, ≥110, ≥120, ≥130, ≥140, ≥150, or ≥160 kDa, for example, 5, 10, 20, 30, 40, 50, 60, 70, 80, 90, or 100 kDa, or a value between any two of the above values. It should be noted that when describing the molecular weight of the conjugated polymer group in a coagulation factor IX fusion protein conjugate, if there are multiple conjugated polymer groups in the conjugate, the sum of the molecular weights of all conjugated polymer groups in the conjugate is calculated, unless otherwise specified.

The polymer used in the present disclosure is known in the prior art, and it can be obtained through a variety of ways, including, for example, commercial routes, or it can be prepared by oneself according to methods known in the art. The present disclosure is not limited to polymers made by any specific method.

After the conjugation reaction, the conjugate can be separated by a suitable method, including, for example, ultrafiltration, dialysis, or chromatography, etc., all of which are within the abilities of those of ordinary skill in the art.

### Pharmaceutical Composition

The coagulation factor IX fusion protein or coagulation factor IX fusion protein conjugate of the present disclosure can have multiple applications, including, for example, for use in prevention and/or treatment of hemorrhagic diseases. Therefore, the present disclosure also provides a pharmaceutical composition for preventing and/or treating hemorrhagic diseases, which comprises a therapeutically effective amount of the fusion protein or conjugate of the present disclosure, and optionally a pharmaceutically acceptable carrier. Preferably, the pharmaceutical composition can be used for the prevention and/or treatment of hemorrhagic diseases, more preferably for the prevention and/or treatment of hemorrhagic diseases in patients with congenital or acquired deficiency of FIX, and spontaneous or surgical bleeding in patients with hemophilia B.

The therapeutically effective amount of the fusion protein or conjugate of the present disclosure depends on the route of administration, the type of subjects, and the physical characteristics of the specific mammal considered. These factors and their relationship with determining the amount are well known to those skilled in the medicinal field. The amount and method of administration can be adjusted to achieve optimal efficacy to deliver the peptide to the subject, but depending on factors well known to those skilled in the medicinal field such as body weight, diet, concomitant medication, and other factors.

The pharmaceutical composition of the present disclosure can be administered in combination therapy, that is, in combination with one or more other agents, wherein the pharmaceutical composition and the other agents are administered together or sequentially. In other embodiments, the other agents may be administered before, during, or after the administration of one or more of the fusion protein, conjugate, or pharmaceutical composition thereof of the present disclosure.

As used herein, “pharmaceutically acceptable carrier” or “physiologically acceptable carrier” can be used interchangeably, including one or more of any and all physiologically compatible salts, solvents, dispersion media, coatings, antibacterial and antifungal agents, isotonic and absorption delaying agents, etc. In some embodiments, the carrier is suitable for intravenous, intramuscular, subcutaneous, parenteral, spinal or epidermal administration (e.g., by injection or infusion). Depending on the route of administration, the therapeutic agent may be coated with certain materials to protect the therapeutic agent from the action of acids and other natural conditions that may inactivate the therapeutic agent.

When administered, the pharmaceutical preparation of the present disclosure is administered in a pharmaceutically acceptable amount in a pharmaceutically acceptable composition. The term “pharmaceutically acceptable” means a non-toxic substance that does not interfere with the biologically active efficacy of the active components. Such preparations generally contain salts, buffers, preservatives, compatible carriers, and optionally other therapeutic agents, such as supplementary immune enhancers, including adjuvants, chemokines and cytokines. When used in medicaments, the salt should be pharmaceutically acceptable. However non-pharmaceutically acceptable salts can be conveniently used to prepare pharmaceutically acceptable salts thereof, thus they are not excluded from the scope of the present disclosure.

If necessary, the coagulation factor IX fusion protein or coagulation factor IX fusion protein conjugate of the present disclosure can be combined with pharmaceutically acceptable carriers. The term “pharmaceutically acceptable carriers” used herein refers to one or more compatible solid or liquid fillers, diluents, or encapsulating substances, which are suitable for administration to mammals such as human. The term “carrier” means organic or inorganic, natural or synthetic components, which are combined with the active components to facilitate application. The components of the pharmaceutical composition can also be blended in a form in which there are no interactions that can significantly disrupt the therapeutic effect of the desired drug.

Preferably, the pharmaceutical composition of the present disclosure may contain a buffer system, and preferably the buffer system is an acetate buffer solution with a pH of about 3.0 to about 6.0, or a phosphate buffer solution with a pH of about 5.0 to about 9.0. In some specific embodiments, suitable buffers include acetate, citrate, borate, and phosphate.

Optionally, the pharmaceutical composition may also contain suitable preservatives, such as benzalkonium chloride, chloro-tert-butanol, parabens and thimerosal.

The pharmaceutical composition can be conveniently presented in a unit dosage form and can be prepared by any known method in the pharmaceutical field. The method comprises the step of combining the active agent with a carrier, which contains one or more accessory ingredients. Generally, the composition is prepared by intimately combining the active compound with one or both of a liquid carrier and a finely divided solid carrier, followed by shaping the product if necessary.

Pharmaceutical compositions suitable for parenteral administration may be sterile aqueous or non-aqueous formulations containing one or more fusion proteins or conjugates. In some embodiments, the formulation is isotonic with the blood of the subject. This preparation can be formulated according to known methods using suitable dispersing or wetting agents and suspending agents. The sterile injection preparation can also be a sterile injection solution or suspension in a non-toxic parenterally acceptable diluent or solvent, for example, a solution in 1,3-butanediol. Acceptable carriers and solvents that can be used include water, Ringer's solution, and isotonic sodium chloride solution. In addition, sterile fixed oils are conventionally used as solvents or suspension media. For this reason, any mild fixed oil can be used, including synthetic mono- or di-glycerides. Also, fatty acids such as oleic acid can be used as the injectable formulations. Carrier formulations suitable for oral, subcutaneous, intravenous, intramuscular, etc. administration can be obtained by referring to reference books in the prior art.

The fusion protein or conjugate of the present disclosure can be prepared with carriers that protect it from rapid release, such as a controlled release formulation, including implants, transdermal patches, and microencapsulated delivery systems. Biodegradable and biocompatible polymers can be used, such as ethylene vinyl acetate, polyanhydrides, polyglycolic acid, collagen, polyorthoesters, and polylactic acid. Many methods for preparing such formulations are known in the prior art.

The pharmaceutical composition of the present disclosure can be administered by any conventional route, including injection or gradual infusion over time. For example, the administration can be oral, intravenous, intraperitoneal, intramuscular, intracavity, intratumor, or transdermal.

The pharmaceutical composition of the present disclosure is administered in an effective amount. An “effective amount” is the amount of any fusion protein or conjugate provided herein, alone or with further doses and/or other therapeutic agents, producing a desired response.

Of course, such an amount will depend on the specific disease to be treated, the severity of the disease, individual patient parameters (including age, physical condition, height and body weight), duration of treatment, the nature of the concurrent treatment (if any), the specific route of administration, and similar factors within the knowledge of medical and health workers. These factors are well known to those skilled in the art, and can be known only by using routine experiments. It is generally preferred to use the maximum dose of each component or combination thereof, that is to say the highest safe dose based on reasonable medical judgment. However, those skilled in the art can understand that patients may require lower doses or allowable doses for medical, psychological, or basically any other reasons.

The pharmaceutical composition used in the foregoing method is preferably sterile and contains an effective amount of the fusion protein or conjugate alone or in combination with another preparation in a weight unit or volume unit suitable for administration to patients to produce the desired response, such as a decrease in blood glucose.

The dosage of the fusion protein or conjugate administered to the subject can be selected according to different parameters, especially according to the mode of administration and the state of the subject. Other factors include the required treatment period. If the response in the subject at the initial dose applied is insufficient, a higher dose (or an effective higher dose achieved by a different, more localized delivery route) within the patient's tolerance can be applied.

In some embodiments, the dosage range of the coagulation factor IX fusion protein and/or coagulation factor IX fusion protein conjugate may be 30 mg/kg of body weight/day to 0.00001 mg/kg of body weight/day, or 3 mg/kg/day to 0.0001 mg/kg/day, or 0.3 mg/kg/day to 0.01 mg/kg/day.

In some embodiments, the pharmaceutical composition of the present disclosure contains 0.20-5 mg/ml of the coagulation factor IX fusion protein and/or 4-40 mg/ml of the coagulation factor IX fusion protein conjugate, preferably 0.20-5mg/ml of the coagulation factor IX fusion protein and/or 4-40 mg/ml of the coagulation factor IX fusion protein conjugate, more preferably 0.5-2 mg/ml of the coagulation factor IX fusion protein and/or 10-20 mg/ml of the coagulation factor IX fusion protein conjugate. Generally, the dosage of the coagulation factor IX fusion protein or coagulation factor IX fusion protein conjugate of the present disclosure can range from about 10 μg/kg of the patient's body weight to about 100,000 μg/kg of the patient's body weight. In some embodiments, the dosage may range from about 0.1 mg/kg to about 20 mg/kg. In some embodiments, the dosage may range from about 0.1 mg/kg to 5 mg/kg, 0.1 mg/kg to 10 mg/kg, or 0.1 mg/kg to 15 mg/kg. In some embodiments, the dosage may range from about 1 mg/kg to 5 mg/kg, 5 mg/kg to 10 mg/kg, 10 mg/kg to 15 mg/kg, or 15 mg/kg to 20 mg/kg. In some embodiments, the dosage is about 0.1 mg/kg, 0.5 mg/kg, 1 mg/kg, 2 mg/kg, 3 mg/kg, 5 mg/kg, 7 mg/kg, 10 mg/kg, 12 mg/kg, 15 mg/kg, 17 mg/kg, 20 mg/kg, 25 mg/kg, or 30 mg/kg. In some embodiments, the dosage is about 1 mg/kg, 3 mg/kg, 5 mg/kg, or 6 mg/kg. Based on the property of the composition, the dose can be delivered continuously (for example, by a continuous pump) or at periodic intervals. In some embodiments, when administered intravenously, the dosage of the coagulation factor IX fusion protein or coagulation factor IX fusion protein conjugate of the present disclosure may be 0.1 to 20 mg/kg or any value therein. The ideal time interval for multiple administrations of a particular composition can be determined by those skilled in the art without undue experimentation. Other dosage regimens of the provided composition are known to those skilled in the art, where the dosage, administration schedule, administration site, administration mode, etc. may be different from the foregoing. In one embodiment, the dosage is administered intravenously. In another embodiment, the dosage regimen is a single intravenous administration.

A kit comprising the coagulation factor IX fusion protein or coagulation factor IX fusion protein conjugate (for example in the pharmaceutical composition) and instructions for use are also within the scope of the present disclosure. The kit may additionally contain at least one of other agents, such as one or more of other agents preventing and/or treating hemorrhagic diseases.

In another embodiment, the kit may include a carrier that is compartmentalized to tightly hold one or more container devices or a series of container devices (such as test tubes, tubes, flasks, bottles, syringes, etc.). The components of the kit can be packaged in an aqueous medium or in a lyophilized form.

The composition may be provided in a lyophilized form or in an aqueous medium.

Preferably, the subject is a vertebrate, more preferably a mammal, most preferably human, but the subject can also be other animals, such as domestic animals (e.g., dogs, cats, etc.), livestock (e.g., cattle, sheep and goats, pigs, horses, etc.) or experimental animals (e.g., monkeys, rats, mice, rabbits, guinea pigs, etc.).

The term “improved circulation half-life” or “extended circulation half-life” means that the molecule of the present disclosure has an altered circulation half-life, preferably an increased circulation half-life compared to the wild-type factor IX molecule. The circulation half-life is preferably increased by at least 10%, preferably at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, more preferably at least 125%, more preferably at least 150%, more preferably at least 175%, more preferably at least 200%, and more preferably at least 250% or 300%. Even more preferably, the molecule has a circulation half-life that is increased by at least 400%, 500%, 600%, or even 700%.

The present disclosure will be further illustrated by the following examples, which should not be construed as further limitations in any way. The entire contents of all references cited in this application (including article references, granted patents, published patent applications and co-pending patent applications) are expressly incorporated herein by reference.

In the following examples, if not specifically noted, the reagents and materials used are commercially available products with at least analytical purity or equivalent levels.

### EXAMPLES

### Example 1 Preparation of Fusion Protein FIX-L1-Fc-L2-P

### 1. Construction of Expression Vector

The FIX fusion proteins constructed in this example have a common molecular structure of FIX-L1-Fc-L2-P, wherein FIX represents a natural coagulation factor IX derived from human, the sequence of which is shown in SEQ ID NO: 1.

L1, representing a first linker between FIX and Fc, is a flexible peptide or a linker peptide composed of a flexible peptide and a rigid structure based on multiple prolines (Pro, P).

In this example, a total of 6 sequences was used as L1, expressed as C1, C2, C3, C4, C5 and GS, respectively, and their specific sequences are as follows:

C1:
(SEQ ID NO: 8)
GGGGSGGGGSGGGGSGGGGSGGGGSVAPPPALPAPVRLPGPA;
C2:
(SEQ ID NO: 9)
GGGGSGGGGSGGGGSGGGGSGGGGSVAPPPALPAVAPPPALPA;
C3:
(SEQ ID NO: 10)
GGGGSGGGGSGGGGSGGGGSGGGGSVAPPPALPAVAPPPALPAVAPPPA
LPAPVRLPGPA
C4:
(SEQ ID NO: 11)
GGGGSGGGGSGGGGSGGGGSGGGGSVAPPPALPAPVRLPGPAVAPPPAL
PAVAPPPALPA;
C5:
(SEQ ID NO: 12)
GGGGSGGGGSGGGGSGGGGSGGGGSVAPPPALPAVAPPPALPAGSVAPP
PALPAVAPPPALPA;
and
GS:
(SEQ ID NO: 13)
GGGGSGGGGSGGGGSGGGGSGGGGS.

Fc is derived from human IgG1 carrying N297A point mutation which has reduced ADCC and CDC activities (see Lund J. et al. Multiple binding sites on the CH2 domain of IgG for mouse Fc gamma R11. Mol Immunol. 1992 January; 29(1):53-9.).

L2 is a second linker, which is mainly used to separate Fc and the Sortase recognition site. Specifically, the sequence of L2 in this example is GSGGSGGGS (SEQ ID NO: 16).

P is an amino acid fragment containing the Sortase recognition site.

Table 1 Summary of all the FIX-L1-Fc-L2-P fusion proteins constructed in this example

TABLE 1

| Protein name | FIX | L1 | Fc | L2 | P |
|---|---|---|---|---|---|
| FIX-C1-Fc-L2-P | SEQ ID NO: 1 | C1 | Human IgG1, | GSGGSGGGGS | LPETGG |
| | | | N297A | | |
| FIX-C2-Fc-L2-P | SEQ ID NO: 1 | C2 | Human IgG1, | GSGGSGGGGS | LPETGG |
| | | | N297A | | |
| FIX-GS-Fc-L2-P | SEQ ID NO: 1 | GS | Human IgG1, | GSGGSGGGGS | LPETGG |
| | | | N297A | | |
| FIX-C3-Fc-L2-P | SEQ ID NO: 1 | C3 | Human IgG1, | GSGGSGGGGS | LPETGG |
| | | | N297A | | |
| FIX-C4-Fc-L2-P | SEQ ID NO: 1 | C4 | Human IgG1, | GSGGSGGGGS | LPETGG |
| | | | N297A | | |
| FIX-C5-Fc-L2-P | SEQ ID NO: 1 | C5 | Human IgG1, | GSGGSGGGGS | LPETGG |
| | | | N297A | | |

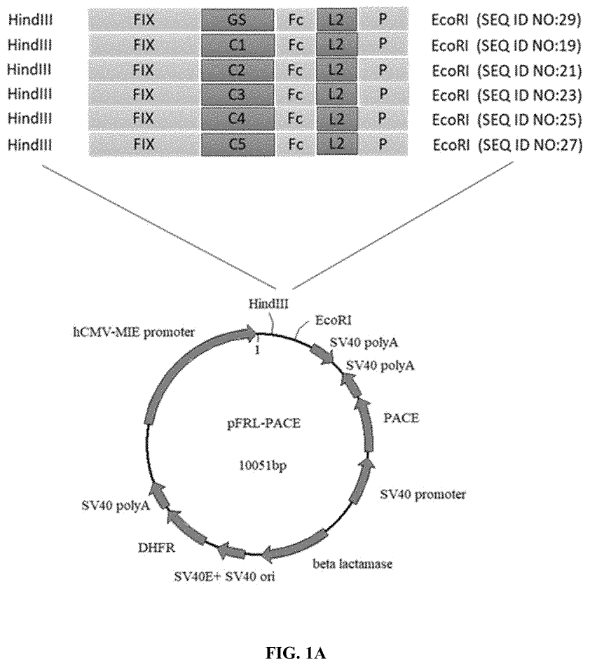

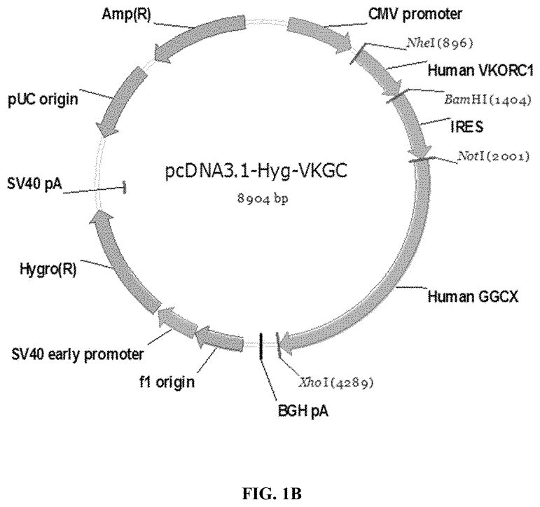

Most of the recombinant FIX expressed in CHO cells exists as a precursor protein, and only about 30% of the propeptide is cleaved from precursor protein to form mature FIX. The processing of mature FIX requires the participation of PACE/furin enzyme, which is a subtilisin-like calcium-dependent serine propeptidase that is located in the trans-Golgi apparatus and can cleave the C-terminus of R46 in the FIX precursor protein to catalyze the conversion of FIX precursor protein into mature FIX. In order to improve the yield and ensure the correct processing of FIX, in this example, a FIX/PACE (Furin) bicistronic expression vector is further constructed, the structure of which is shown in FIG. 1A.

### Construction of FIX/PACE (Furin) Bicistronic Expression Vector

### a. Construction of Expression Vector pFRL-rPS Expressing PACE

The sequence of PACE/Furin refers to GenBank accession number NP_002560.1. The coding sequence of PACE/Furin amino acids 1-715 (SEQ ID NO: 17) was first codon-optimized for the expression host. After full gene synthesis, the sequence was cloned into pcDNA3.1 (+) vector, and the obtained plasmid was named pcDNA3.1-P-SOL (Shanghai Generay Biotech Co., Ltd.). A complete gene expression cassette including the promoter SV40, PACE/Furin gene and Poly A was obtained through PCR amplification with primers F1 and R1 (see below for the sequence) using the plasmid pcDNA3.1-P-SOL as a template. By the BglII restriction sites at both ends, the fragment was inserted into the pFRL-DHFR vector that was single digested with BamHI. The positive clones were selected and verified by sequencing, and the vector was named pFRL-rPS.

F1:
(SEQ ID NO: 30)
5′-TCAGGAAGATCTCGCGAATTAATTCTGTGGAATGTGT-3′;
R1:
(SEQ ID NO: 31)
5′-TATCCTAGATCTGCTGGCACGACAGGTTTCCCGAC T-3′.

The sequence of PACE/Furin amino acid 1-715 is as follow:

(SEQ ID NO: 17)
MELRPWLLWVVAATGTLVLLAADAQGQKVFTNTWAVRIPGGPAVANSVA
RKHGFLNLGQIFGDYYHFWHRGVTKRSLSPHRPRHSRLQREPQVQWLEQ
QVAKRRTKRDVYQEPTDPKFPQQWYLSGVTQRDLNVKAAWAQGYTGHGI
VVSILDDGIEKNHPDLAGNYDPGASFDVNDQDPDPQPRYTQMNDNRHGT
RCAGEVAAVANNGVCGVGVAYNARIGGVRWILDGEVTDAVEARSLGLNP
NHIHIYSASWGPEDDGKTVDGPARLAEEAFFRGVSQGRGGLGSIFVWAS
GNGGREHDSCNCDGYTNSIYTLSISSATQFGNVPWYSEACSSTLATTYS
SGNQNEKQIVTTDLRQKCTESHTGTSASAPLAAGIIALTLEANKNLTWR
DMQHLVVQTSKPAHLNANDWATNGVGRKVSHSYGYGLLDAGAMVALAQN
WTTVAPQRKCIIDILTEPKDIGKRLEVRKTVTACLGEPNHITRLEHAQA
RLTLSYNRRGDLAIHLVSPMGTRSTLLAARPHDYSADGFNDWAFMTTHS
WDEDPSGEWVLEIENTSEANNYGTLTKFTLVLYGTAPEGLPVPPESSGC
KTLTSSQACVVCEEGFSLHQKSCVQHCPPGFAPQVLDTHYSTENDVETI
RASVCAPCHASCATCQGPALTDCLSCPSHASLDPVEQTCSRQSQSSRES
PPQQQPPRLPPEVEAGQRLRAGLLPSHLPE.

### b. Construction of Bicistronic Expression Vectors Expressing FIX-L1-Fc-L2-P

### b1) pFRL-rPS-FIX-C1-Fc-L2-P

The nucleic acid sequence encoding FIX-C1-Fc-L2-P (that is, the sequence of L1 in FIX-L1-Fc-L2-P is C1, and so on) was codon-optimized (the amino acid sequence is shown in SEQ ID NO: 18, and the codon-optimized nucleic acid coding sequence is shown in SEQ ID NO: 19). After full gene synthesis by Shanghai Generay Biotech Co., Ltd., the fragment was inserted into pFRL-rPS through the HindIII/EcoRI double restriction site to construct the bicistronic expression vector pFRL-rPS-FIX-C1-Fc-L2-P co-expressing FIX-C1-Fc-L2-P/Furin.

Amino acid sequence of FIX-C1-Fc-L2-P:
(SEQ ID NO: 18)
MQRVNMIMAESPGLITICLLGYLLSAECTVFLDHENANKILNRPKRYNS
GKLEEFVQGNLERECMEEKCSFEEAREVFENTERTTEFWKQYVDGDQCE
SNPCLNGGSCKDDINSYECWCPFGFEGKNCELDVTCNIKNGRCEQFCKN
SADNKVVCSCTEGYRLAENQKSCEPAVPFPCGRVSVSQTSKLTRAETVF
PDVDYVNSTEAETILDNITQSTQSFNDFTRVVGGEDAKPGQFPWQVVLN
GKVDAFCGGSIVNEKWIVTAAHCVETGVKITVVAGEHNIEETEHTEQKR
NVIRIIPHENYNAAINKYNHDIALLELDEPLVLNSYVTPICIADKEYTN
IFLKFGSGYVSGWGRVFHKGRSALVLQYLRVPLVDRATCLRSTKFTIYN
NMFCAGFHEGGRDSCQGDSGGPHVTEVEGTSFLTGIISWGEECAMKGKY
GIYTKVSRYVNWIKEKTKLTGGGGSGGGGSGGGGSGGGGSGGGGSVAPP
PALPAPVRLPGPADKTHTCPPCPAPELLGGPSVFLFPPKPKDTLMISRT
PEVTCVVVDVSHEDPEVKFNWYVDGVEVHNAKTKPREEQYASTYRVVSV
LTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGS
FFLYSKLTVDKSRWQQGNVFScSVMHEALHNHYTQKSLSLSPGKGSGGS
GGGGSLPETGG.
Nucleotide sequence of FIX-C1-Fc-L2-P:
(SEQ ID NO: 19)
ATGCAGCGCGTCAATATGATTATGGCCGAGTCTCCAGGCCTCATTACCA
TTTGTCTGCTGGGCTACCTGCTGTCCGCAGAGTGTACCGTCTTCCTCGA
CCACGAGAACGCAAATAAGATTCTCAATAGGCCTAAGCGCTACAATTCC
GGAAAGCTCGAAGAGTTTGTCCAGGGAAATCTGGAGCGCGAGTGTATGG
AGGAAAAGTGTAGCTTTGAAGAAGCTAGAGAGGTGTTTGAGAATACTGA
GCGCACTACTGAGTTTTGGAAGCAGTATGTGGATGGCGATCAGTGCGAG
TCCAATCCATGCCTGAATGGCGGATCTTGCAAGGATGATATTAATTCTT
ACGAGTGTTGGTGCCCATTCGGCTTTGAGGGAAAGAATTGCGAACTGGA
TGTCACATGTAACATCAAGAACGGCAGATGCGAGCAGTTTTGTAAGAAC
AGCGCCGATAATAAGGTGGTGTGTAGTTGTACTGAGGGATACCGCCTCG
CAGAGAATCAGAAGTCCTGTGAGCCCGCTGTGCCATTTCCTTGTGGCCG
GGTCAGCGTCAGCCAGACTTCTAAGCTGACACGCGCCGAGACCGTCTTT
CCAGATGTGGATTATGTCAACTCTACTGAGGCCGAGACCATTCTCGATA
ATATTACTCAGTCTACACAGTCCTTTAACGACTTCACTCGGGTGGTGGG
CGGAGAGGACGCAAAGCCTGGCCAGTTTCCTTGGCAGGTCGTGCTCAAC
GGAAAGGTGGACGCATTTTGTGGCGGATCTATCGTGAATGAGAAGTGGA
TTGTGACCGCCGCTCACTGCGTCGAGACAGGCGTCAAGATTACCGTGGT
CGCTGGAGAGCACAACATTGAGGAAACAGAGCACACCGAGCAGAAGCGC
AATGTGATTCGCATCATCCCACACCACAACTACAACGCCGCTATTAATA
AGTACAACCACGACATCGCCCTGTTAGAACTAGATGAGCCTCTGGTCCT
GAATAGTTACGTCACTCCTATTTGTATCGCCGATAAAGAATACACTAAC
ATCTTTCTCAAGTTCGGCTCTGGCTATGTATCTGGATGGGGACGGGTGT
TTCACAAGGGACGGTCCGCACTCGTGCTACAATACCTCCGGGTGCCACT
GGTGGATCGGGCAACATGCCTCCGGTCTACCAAGTTTACTATATACAAT
AACATGTTTTGCGCCGGCTTTCACGAGGGCGGACGGGATAGTTGCCAGG
GCGATAGTGGAGGCCCTCATGTCACAGAGGTGGAGGGAACTAGCTTTCT
GACAGGCATTATCTCTTGGGGCGAAGAGTGCGCTATGAAGGGAAAGTAC
GGCATCTATACTAAGGTCTCGCGTTACGTCAATTGGATCAAGGAAAAGA
CAAAGCTGACAGGCGGCGGAGGGAGCGGCGGCGGAGGCTCTGGCGGCGG
CGGATCTGGCGGAGGCGGCTCTGGCGGAGGCGGATCCGTCGCTCCACCA
CCTGCACTCCCCGCCCCTGTCAGACTGCCTGGACCCGCTGATAAGACCC
ACACATGTCCACCTTGCCCAGCACCTGAGCTGCTGGGCGGACCTAGCGT
GTTTCTGTTCCCACCAAAGCCAAAGGACACCCTCATGATTAGTAGAACC
CCAGAGGTGACTTGCGTCGTGGTCGATGTCAGTCACGAGGACCCCGAGG
TGAAGTTTAATTGGTATGTAGATGGCGTCGAGGTGCACAACGCTAAGAC
CAAGCCACGCGAAGAGCAGTACGCATCTACTTACCGGGTCGTCTCTGTG
CTGACTGTGCTGCACCAGGATTGGCTCAATGGCAAAGAGTACAAGTGTA
AGGTCAGTAATAAGGCCCTCCCCGCACCTATTGAAAAGACTATTAGCAA
GGCAAAGGGCCAGCCTAGGGAGCCCCAGGTCTACACCCTGCCCCCTTCC
CGCGATGAGCTGACTAAGAATCAGGTCTCTCTCACTTGCCTCGTGAAGG
GCTTTTACCCATCCGACATTGCCGTGGAGTGGGAGTCCAACGGACAGCC
AGAGAATAACTACAAGACAACTCCTCCCGTGCTCGATAGCGACGGTTCA
TTCTTCCTGTACTCCAAGCTCACCGTTGATAAGTCTCGGTGGCAGCAGG
GAAATGTGTTCTCTTGTTCCGTCATGCACGAGGCACTGCACAATCACTA
CACCCAGAAGTCCCTGTCCCTCAGCCCTGGCAAAGGCAGCGGCGGATCT
GGAGGCGGCGGATCCCTCCCAGAGACCGGCGGATGA.

### b2) pFRL-rPS-FIX-C2-Fc-L2-P

Using the coding sequence of FIX-C1-Fc-L2-P as a template, C2 sequence was added to the 5′ end of the primer, and the Fc-L2-P sequence was amplified by PCR to obtain the C2-Fc-L2-P sequence. The sequences of primers F2 and R2 used for amplification are as follows:

Forward primer F2:
(SEQ ID NO: 32)
5′-TTCGCGGATCCGTCGCTCCACCACCTGCACTCCCCGCCGTGGCTCC
TCCACCTGCTTTGCCCGCTGATAAGACCCACACATGTCCAC-3′,
Reverse primer R2:
(SEQ ID NO: 33)
5′-TAACCGGAATTCATTATCCGCCGGTCTCTGGGAGAGATCCGCCGCC
TCCAGATCCGCCGCTGCCGCCAGGGCTGAGGGACAGGGACT-3′.

Next, through the BamHI/EcoRI double restriction site, the C1-Fc-L2-P sequence in the vector pFRL-rPS-FIX-C1-Fc-L2-P was replaced with the amplified product C2-Fc-L2-P, thereby obtaining the bicistronic expression vector pFRL-rPS-FIX-C2-Fc-L2-P co-expressing FIX-C2-Fc-L2-P/Furin.

Amino acid sequence of FIX-C2-Fc-L2-P:
(SEQ ID NO: 20)
MQRVNMIMAESPGLITICLLGYLLSAECTVFLDHENANKILNRPKRYNS
GKLEEFVQGNLERECMEEKCSFEEAREVFENTERTTEFWKQYVDGDQCE
SNPCLNGGSCKDDINSYECWCPFGFEGKNCELDVTCNIKNGRCEQFCKN
SADNKVVCSCTEGYRLAENQKSCEPAVPFPCGRVSVSQTSKLTRAETVF
PDVDYVNSTEAETILDNITQSTQSENDFTRVVGGEDAKPGQFPWQVVLN
GKVDAFCGGSIVNEKWIVTAAHCVETGVKITVVAGEHNIEETEHTEQKR
NVIRIIPHEINYNAAINKYNHDIALLELDEPLVLNSYVTPICIADKEYT
NIFLKEGSGYVSGWGRVFHKGRSALVLQYLRVPLVDRATCLRSTKFTIY
NNMFCAGFHEGGRDSCQGDSGGPHVTEVEGTSFLTGIISWGEECAMKGK
YGIYTKVSRYVNWIKEKTKLTGGGGSGGGGSGGGGSGGGGSGGGGSVAP
PPALPAVAPPPALPADKTHTCPPCPAPELLGGPSVFLEPPKPKDTLMIS
RTPEVTCVVVDVSHEDPEVKFNWYVDGVEVHNAKTKPREEQYASTYRVV
SVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGGSGG
SGGGGSLPETGG.
Nucleotide sequence of FIX-C2-Fc-L2-P:
(SEQ ID NO: 21)
ATGCAGCGCGTCAATATGATTATGGCCGAGTCTCCAGGCCTCATTACCA
TTTGTCTGCTGGGCTACCTGCTGTCCGCAGAGTGTACCGTCTTCCTCGA
CCACGAGAACGCAAATAAGATTCTCAATAGGCCTAAGCGCTACAATTCC
GGAAAGCTCGAAGAGTTTGTCCAGGGAAATCTGGAGCGCGAGTGTATGG
AGGAAAAGTGTAGCTTTGAAGAAGCTAGAGAGGTGTTTGAGAATACTGA
GCGCACTACTGAGTTTTGGAAGCAGTATGTGGATGGCGATCAGTGCGAG
TCCAATCCATGCCTGAATGGCGGATCTTGCAAGGATGATATTAATTCTT
ACGAGTGTTGGTGCCCATTCGGCTTTGAGGGAAAGAATTGCGAACTGGA
TGTCACATGTAACATCAAGAACGGCAGATGCGAGCAGTTTTGTAAGAAC
AGCGCCGATAATAAGGTGGTGTGTAGTTGTACTGAGGGATACCGCCTCG
CAGAGAATCAGAAGTCCTGTGAGCCCGCTGTGCCATTTCCTTGTGGCCG
GGTCAGCGTCAGCCAGACTTCTAAGCTGACACGCGCCGAGACCGTCTTT
CCAGATGTGGATTATGTCAACTCTACTGAGGCCGAGACCATTCTCGATA
ATATTACTCAGTCTACACAGTCCTTTAACGACTTCACTCGGGTGGTGGG
CGGAGAGGACGCAAAGCCTGGCCAGTTTCCTTGGCAGGTCGTGCTCAAC
GGAAAGGTGGACGCATTTTGTGGCGGATCTATCGTGAATGAGAAGTGGA
TTGTGACCGCCGCTCACTGCGTCGAGACAGGCGTCAAGATTACCGTGGT
CGCTGGAGAGCACAACATTGAGGAAACAGAGCACACCGAGCAGAAGCGC
AATGTGATTCGCATCATCCCACACCACAACTACAACGCCGCTATTAATA
AGTACAACCACGACATCGCCCTGTTAGAACTAGATGAGCCTCTGGTCCT
GAATAGTTACGTCACTCCTATTTGTATCGCCGATAAAGAATACACTAAC
ATCTTTCTCAAGTTCGGCTCTGGCTATGTATCTGGATGGGGACGGGTGT
TTCACAAGGGACGGTCCGCACTCGTGCTACAATACCTCCGGGTGCCACT
GGTGGATCGGGCAACATGCCTCCGGTCTACCAAGTTTACTATATACAAT
AACATGTTTTGCGCCGGCTTTCACGAGGGCGGACGGGATAGTTGCCAGG
GCGATAGTGGAGGCCCTCATGTCACAGAGGTGGAGGGAACTAGCTTTCT
GACAGGCATTATCTCTTGGGGCGAAGAGTGCGCTATGAAGGGAAAGTAC
GGCATCTATACTAAGGTCTCGCGTTACGTCAATTGGATCAAGGAAAAGA
CAAAGCTGACAGGCGGCGGAGGGAGCGGCGGCGGAGGCTCTGGCGGCGG
CGGATCTGGCGGAGGCGGCTCTGGCGGAGGCGGATCCGTCGCTCCACCA
CCTGCACTCCCCGCCGTGGCTCCTCCACCTGCTTTGCCCGCTGATAAGA
CCCACACATGTCCACCTTGCCCAGCACCTGAGCTGCTGGGCGGACCTAG
CGTGTTTCTGTTCCCACCAAAGCCAAAGGACACCCTCATGATTAGTAGA
ACCCCAGAGGTGACTTGCGTCGTGGTCGATGTCAGTCACGAGGACCCCG
AGGTGAAGTTTAATTGGTATGTAGATGGCGTCGAGGTGCACAACGCTAA
GACCAAGCCACGCGAAGAGCAGTACGCATCTACTTACCGGGTCGTCTCT
GTGCTGACTGTGCTGCACCAGGATTGGCTCAATGGCAAAGAGTACAAGT
GTAAGGTCAGTAATAAGGCCCTCCCCGCACCTATTGAAAAGACTATTAG
CAAGGCAAAGGGCCAGCCTAGGGAGCCCCAGGTCTACACCCTGCCCCCT
TCCCGCGATGAGCTGACTAAGAATCAGGTCTCTCTCACTTGCCTCGTGA
AGGGCTTTTACCCATCCGACATTGCCGTGGAGTGGGAGTCCAACGGACA
GCCAGAGAATAACTACAAGACAACTCCTCCCGTGCTCGATAGCGACGGT
TCATTCTTCCTGTACTCCAAGCTCACCGTTGATAAGTCTCGGTGGCAGC
AGGGAAATGTGTTCTCTTGTTCCGTCATGCACGAGGCACTGCACAATCA
CTACACCCAGAAGTCCCTGTCCCTCAGCCCTGGCGGCAGCGGCGGATCT
GGAGGCGGCGGATCTCTCCCAGAGACCGGCGGATAA.

### b3) Construction of pFRL-rPS-FIX-C3-Fc-L2-P

The vector construction method is the same as for pFRL-rPS-FIX-C2-Fc-L2-P. The PCR primers for amplifying the coding sequence of C3-Fc-L2-P include forward primers F3: 5′-TGGCTCCTCCACCTGCTTTGCCCGCTGTCGCTCCACCACCTGCACTCC-3′ (SEQ ID NO: 34) and F4: 5′-TAGATCTGTCGCTCCACCACCTGCACTCCC CGCCGTGGCTCCTCCACCTGCTTTGCC-3′ (SEQ ID NO: 35), and reverse primer R3: 5′-TGAATTCT TATCATCCGCCGGTCTCTGGGAG-3′ (SEQ ID NO: 36). The C3-Fc-L2-P sequence was obtained by 2 rounds of PCR amplification.

Amino acid sequence of FIX-C3-Fc-L2-P:
(SEQ ID NO: 22)
MQRVNMIMAESPGLITICLLGYLLSAECTVFLDHENANKILNRPKRYNS
GKLEEFVQGNLERECMEEKCSFEEAREVFENTERTTEFWKQYVDGDQCE
SNPCLNGGSCKDDINSYECWCPFGFEGKNCELDVTCNIKNGRCEQFCKN
SADNKVVCSCTEGYRLAENQKSCEPAVPFPCGRVSVSQTSKLTRAETVF
PDVDYVNSTEAETILDNITQSTQSENDFTRVVGGEDAKPGQFPWQVVLN
GKVDAFCGGSIVNEKWIVTAAHCVETGVKITVVAGEHNIEETEHTEQKR
NVIRIIPHEINYNAAINKYNHDIALLELDEPLVLNSYVTPICIADKEYT
NIFLKEGSGYVSGWGRVFHKGRSALVLQYLRVPLVDRATCLRSTKFTIY
NNMFCAGFHEGGRDSCQGDSGGPHVTEVEGTSFLTGIISWGEECAMKGK
YGIYTKVSRYVNWIKEKTKLTGGGGSGGGGSGGGGSGGGGSGGGGSVAP
PPALPAVAPPPALPAVAPPPALPAPVRLPGPADKTHTCPPCPAPELLGG
PSVFLEPPKPKDTLMISRTPEVTCVVVDVSHEDPEVKFNWYVDGVEVHN
AKTKPREEQYASTYRVVSVLTVLHQDWLNGKEYKCKVSNKALPAPIEKT
ISKAKGQPREPQVYTLPPSRDELTKNQVSLTCLVKGEYPSDIAVEWESN
GQPENNYKTTPPVLDSDGSFFLYSKLTVDKSRWQQGNVFSCSVMHEALH
NHYTQKSLSLSPGKGSGGSGGGGSLPETGG.
Nucleotide sequence of FIX-C3-Fc-L2-P:
(SEQ ID NO: 23)
ATGCAGCGCGTCAATATGATTATGGCCGAGTCTCCAGGCCTCATTACCA
TTTGTCTGCTGGGCTACCTGCTGTCCGCAGAGTGTACCGTCTTCCTCGA
CCACGAGAACGCAAATAAGATTCTCAATAGGCCTAAGCGCTACAATTCC
GGAAAGCTCGAAGAGTTTGTCCAGGGAAATCTGGAGCGCGAGTGTATGG
AGGAAAAGTGTAGCTTTGAAGAAGCTAGAGAGGTGTTTGAGAATACTGA
GCGCACTACTGAGTTTTGGAAGCAGTATGTGGATGGCGATCAGTGCGAG
TCCAATCCATGCCTGAATGGCGGATCTTGCAAGGATGATATTAATTCTT
ACGAGTGTTGGTGCCCATTCGGCTTTGAGGGAAAGAATTGCGAACTGGA
TGTCACATGTAACATCAAGAACGGCAGATGCGAGCAGTTTTGTAAGAAC
AGCGCCGATAATAAGGTGGTGTGTAGTTGTACTGAGGGATACCGCCTCG
CAGAGAATCAGAAGTCCTGTGAGCCCGCTGTGCCATTTCCTTGTGGCCG
GGTCAGCGTCAGCCAGACTTCTAAGCTGACACGCGCCGAGACCGTCTTT
CCAGATGTGGATTATGTCAACTCTACTGAGGCCGAGACCATTCTCGATA
ATATTACTCAGTCTACACAGTCCTTTAACGACTTCACTCGGGTGGTGGG
CGGAGAGGACGCAAAGCCTGGCCAGTTTCCTTGGCAGGTCGTGCTCAAC
GGAAAGGTGGACGCATTTTGTGGCGGATCTATCGTGAATGAGAAGTGGA
TTGTGACCGCCGCTCACTGCGTCGAGACAGGCGTCAAGATTACCGTGGT
CGCTGGAGAGCACAACATTGAGGAAACAGAGCACACCGAGCAGAAGCGC
AATGTGATTCGCATCATCCCACACCACAACTACAACGCCGCTATTAATA
AGTACAACCACGACATCGCCCTGTTAGAACTAGATGAGCCTCTGGTCCT
GAATAGTTACGTCACTCCTATTTGTATCGCCGATAAAGAATACACTAAC
ATCTTTCTCAAGTTCGGCTCTGGCTATGTATCTGGATGGGGACGGGTGT
TTCACAAGGGACGGTCCGCACTCGTGCTACAATACCTCCGGGTGCCACT
GGTGGATCGGGCAACATGCCTCCGGTCTACCAAGTTTACTATATACAAT
AACATGTTTTGCGCCGGCTTTCACGAGGGCGGACGGGATAGTTGCCAGG
GCGATAGTGGAGGCCCTCATGTCACAGAGGTGGAGGGAACTAGCTTTCT
GACAGGCATTATCTCTTGGGGCGAAGAGTGCGCTATGAAGGGAAAGTAC
GGCATCTATACTAAGGTCTCGCGTTACGTCAATTGGATCAAGGAAAAGA
CAAAGCTGACAGGCGGCGGAGGGAGCGGCGGCGGAGGCTCTGGCGGCGG
CGGATCTGGCGGAGGCGGCTCTGGCGGAGGCGGATCTGTCGCTCCACCA
CCTGCACTCCCCGCCGTGGCTCCTCCACCTGCTTTGCCCGCTGTCGCTC
CACCACCTGCACTCCCCGCCCCTGTCAGACTGCCTGGACCCGCTGATAA
GACCCACACATGTCCACCTTGCCCAGCACCTGAGCTGCTGGGCGGACCT
AGCGTGTTTCTGTTCCCACCAAAGCCAAAGGACACCCTCATGATTAGTA
GAACCCCAGAGGTGACTTGCGTCGTGGTCGATGTCAGTCACGAGGACCC
CGAGGTGAAGTTTAATTGGTATGTAGATGGCGTCGAGGTGCACAACGCT
AAGACCAAGCCACGCGAAGAGCAGTACGCATCTACTTACCGGGTCGTCT
CTGTGCTGACTGTGCTGCACCAGGATTGGCTCAATGGCAAAGAGTACAA
GTGTAAGGTCAGTAATAAGGCCCTCCCCGCACCTATTGAAAAGACTATT
AGCAAGGCAAAGGGCCAGCCTAGGGAGCCCCAGGTCTACACCCTGCCCC
CTTCCCGCGATGAGCTGACTAAGAATCAGGTCTCTCTCACTTGCCTCGT
GAAGGGCTTTTACCCATCCGACATTGCCGTGGAGTGGGAGTCCAACGGA
CAGCCAGAGAATAACTACAAGACAACTCCTCCCGTGCTCGATAGCGACG
GTTCATTCTTCCTGTACTCCAAGCTCACCGTTGATAAGTCTCGGTGGCA
GCAGGGAAATGTGTTCTCTTGTTCCGTCATGCACGAGGCACTGCACAAT
CACTACACCCAGAAGTCCCTGTCCCTCAGCCCTGGCAAAGGCAGCGGCG
GATCTGGAGGCGGCGGATCCCTCCCAGAGACCGGCGGATGA.

### b4) Construction of pFRL-rPS-FIX-C4-Fc-L2-P

The construction method is the same as for pFRL-rPS-FIX-C2-Fc-P. The PCR primers for amplifying the C4-Fc-L2-P gene include forward primers F5: 5′-CCCTGT CAGACTGCCTGGACCCGCTGTCGCTCCACCACCTGCACTCC-3′ (SEQ ID NO: 37) and F6: 5′-TAGATCTGTCGCTCCACCACCTGCACTCCCCGCCCCTGTCAGACTGCCTGGACC-3′ (SEQ ID NO: 38), and reverse primer R3. The C4-Fc-L2-P sequence was obtained by 2 rounds of PCR amplification.

Amino acid sequence of FIX-C4-Fc-L2-P:
(SEQ ID NO: 24)
MQRVNMIMAESPGLITICLLGYLLSAECTVFLDHENANKILNRPKRYNS
GKLEEFVQGNLERECMEEKCSFEEAREVFENTERTTEFWKQYVDGDQCE
SNPCLNGGSCKDDINSYECWCPFGFEGKNCELDVTCNIKNGRCEQFCKN
SADNKVVCSCTEGYRLAENQKSCEPAVPFPCGRVSVSQTSKLTRAETVF
PDVDYVNSTEAETILDNITQSTQSENDFTRVVGGEDAKPGQFPWQVVLN
GKVDAFCGGSIVNEKWIVTAAHCVETGVKITVVAGEHNIEETEHTEQKR
NVIRIIPHEINYNAAINKYNHDIALLELDEPLVLNSYVTPICIADKEYT
NIFLKEGSGYVSGWGRVFHKGRSALVLQYLRVPLVDRATCLRSTKFTIY
NNMFCAGFHEGGRDSCQGDSGGPHVTEVEGTSFLTGIISWGEECAMKGK
YGIYTKVSRYVNWIKEKTKLTGGGGSGGGGSGGGGSGGGGSGGGGSVAP
PPALPAPVRLPGPAVAPPPALPAVAPPPALPADKTHTCPPCPAPELLGG
PSVFLFPPKPKDTLMISRTPEVTCVVVDVSHEDPEVKFNWYVDGVEVHN
AKTKPREEQYASTYRVVSVLTVLHQDWLNGKEYKCKVSNKALPAPIEKT
ISKAKGQPREPQVYTLPPSRDELTKNQVSLTCLVKGEYPSDIAVEWESN
GQPENNYKTTPPVLDSDGSFFLYSKLTVDKSRWQQGNVFSCSVMHEALH
NHYTQKSLSLSPGGSGGSGGGGSLPETGG.
Nucleotide sequence of FIX-C4-Fc-L2-P
(SEQ ID NO: 25)
ATGCAGCGCGTCAATATGATTATGGCCGAGTCTCCAGGCCTCATTACCA
TTTGTCTGCTGGGCTACCTGCTGTCCGCAGAGTGTACCGTCTTCCTCGA
CCACGAGAACGCAAATAAGATTCTCAATAGGCCTAAGCGCTACAATTCC
GGAAAGCTCGAAGAGTTTGTCCAGGGAAATCTGGAGCGCGAGTGTATGG
AGGAAAAGTGTAGCTTTGAAGAAGCTAGAGAGGTGTTTGAGAATACTGA
GCGCACTACTGAGTTTTGGAAGCAGTATGTGGATGGCGATCAGTGCGAG
TCCAATCCATGCCTGAATGGCGGATCTTGCAAGGATGATATTAATTCTT
ACGAGTGTTGGTGCCCATTCGGCTTTGAGGGAAAGAATTGCGAACTGGA
TGTCACATGTAACATCAAGAACGGCAGATGCGAGCAGTTTTGTAAGAAC
AGCGCCGATAATAAGGTGGTGTGTAGTTGTACTGAGGGATACCGCCTCG
CAGAGAATCAGAAGTCCTGTGAGCCCGCTGTGCCATTTCCTTGTGGCCG
GGTCAGCGTCAGCCAGACTTCTAAGCTGACACGCGCCGAGACCGTCTTT
CCAGATGTGGATTATGTCAACTCTACTGAGGCCGAGACCATTCTCGATA
ATATTACTCAGTCTACACAGTCCTTTAACGACTTCACTCGGGTGGTGGG
CGGAGAGGACGCAAAGCCTGGCCAGTTTCCTTGGCAGGTCGTGCTCAAC
GGAAAGGTGGACGCATTTTGTGGCGGATCTATCGTGAATGAGAAGTGGA
TTGTGACCGCCGCTCACTGCGTCGAGACAGGCGTCAAGATTACCGTGGT
CGCTGGAGAGCACAACATTGAGGAAACAGAGCACACCGAGCAGAAGCGC
AATGTGATTCGCATCATCCCACACCACAACTACAACGCCGCTATTAATA
AGTACAACCACGACATCGCCCTGTTAGAACTAGATGAGCCTCTGGTCCT
GAATAGTTACGTCACTCCTATTTGTATCGCCGATAAAGAATACACTAAC
ATCTTTCTCAAGTTCGGCTCTGGCTATGTATCTGGATGGGGACGGGTGT
TTCACAAGGGACGGTCCGCACTCGTGCTACAATACCTCCGGGTGCCACT
GGTGGATCGGGCAACATGCCTCCGGTCTACCAAGTTTACTATATACAAT
AACATGTTTTGCGCCGGCTTTCACGAGGGCGGACGGGATAGTTGCCAGG
GCGATAGTGGAGGCCCTCATGTCACAGAGGTGGAGGGAACTAGCTTTCT
GACAGGCATTATCTCTTGGGGCGAAGAGTGCGCTATGAAGGGAAAGTAC
GGCATCTATACTAAGGTCTCGCGTTACGTCAATTGGATCAAGGAAAAGA
CAAAGCTGACAGGCGGCGGAGGGAGCGGCGGCGGAGGCTCTGGCGGCGG
CGGATCTGGCGGAGGCGGCTCTGGCGGAGGCGGATCTGTCGCTCCACCA
CCTGCACTCCCCGCCCCTGTCAGACTGCCTGGACCCGCTGTCGCTCCAC
CACCTGCACTCCCCGCCGTGGCTCCTCCACCTGCTTTGCCCGCTGATAA
GACCCACACATGTCCACCTTGCCCAGCACCTGAGCTGCTGGGCGGACCT
AGCGTGTTTCTGTTCCCACCAAAGCCAAAGGACACCCTCATGATTAGTA
GAACCCCAGAGGTGACTTGCGTCGTGGTCGATGTCAGTCACGAGGACCC
CGAGGTGAAGTTTAATTGGTATGTAGATGGCGTCGAGGTGCACAACGCT
AAGACCAAGCCACGCGAAGAGCAGTACGCATCTACTTACCGGGTCGTCT
CTGTGCTGACTGTGCTGCACCAGGATTGGCTCAATGGCAAAGAGTACAA
GTGTAAGGTCAGTAATAAGGCCCTCCCCGCACCTATTGAAAAGACTATT
AGCAAGGCAAAGGGCCAGCCTAGGGAGCCCCAGGTCTACACCCTGCCCC
CTTCCCGCGATGAGCTGACTAAGAATCAGGTCTCTCTCACTTGCCTCGT
GAAGGGCTTTTACCCATCCGACATTGCCGTGGAGTGGGAGTCCAACGGA
CAGCCAGAGAATAACTACAAGACAACTCCTCCCGTGCTCGATAGCGACG
GTTCATTCTTCCTGTACTCCAAGCTCACCGTTGATAAGTCTCGGTGGCA
GCAGGGAAATGTGTTCTCTTGTTCCGTCATGCACGAGGCACTGCACAAT
CACTACACCCAGAAGTCCCTGTCCCTCAGCCCTGGCGGCAGCGGCGGAT
CTGGAGGCGGCGGATCTCTCCCAGAGACCGGCGGATGA.

### b5) Construction of pFRL-rPS-FIX-05-Fc-L2-P

Using FIX-C2-Fc-L2-P as a template, PCR amplification of FIX-C2 fragment was performed (forward primer F7: 5′-ATATAAGCTTCCGCCACCATGCAGCGC-3′ (SEQ ID NO: 39) and reverse primer R4: 5′-ATATGGATCCAGCGGGCAAAGCAGGTGG AGGAGC-3′ (SEQ ID NO: 40)). The obtained fragment was cloned into the vector pFRL-rPS-FIX-C2-Fc-L2-P through the HindIII/BamHI double restriction site to generate the bicistronic expression vector pFRL-rPS-FIX-C5-Fc-L2-P co-expressing FIX-C5-Fc-L2-P/Furin (C5 is actually a tandem of two C2).

Amino acid sequence of FIX-C5-Fc-L2-P:
(SEQ ID NO: 26)
MQRVNMIMAESPGLITICLLGYLLSAECTVFLDHENANKILNRPKRYNS
GKLEEFVQGNLERECMEEKCSFEEAREVFENTERTTEFWKQYVDGDQCE
SNPCLNGGSCKDDINSYECWCPFGFEGKNCELDVTCNIKNGRCEQFCKN
SADNKVVCSCTEGYRLAENQKSCEPAVPFPCGRVSVSQTSKLTRAETVF
PDVDYVNSTEAETILDNITQSTQSENDFTRVVGGEDAKPGQFPWQVVLN
GKVDAFCGGSIVNEKWIVTAAHCVETGVKITVVAGEHNIEETEHTEQKR
NVIRIIPHEINYNAAINKYNHDIALLELDEPLVLNSYVTPICIADKEYT
NIFLKEGSGYVSGWGRVFHKGRSALVLQYLRVPLVDRATCLRSTKFTIY
NNMFCAGFHEGGRDSCQGDSGGPHVTEVEGTSFLTGIISWGEECAMKGK
YGIYTKVSRYVNWIKEKTKLTGGGGSGGGGSGGGGSGGGGSGGGGSVAP
PPALPAVAPPPALPAGSVAPPPALPAVAPPPALPADKTHTCPPCPAPEL
LGGPSVFLEPPKPKDTLMISRTPEVTCVVVDVSHEDPEVKFNWYVDGVE
VHNAKTKPREEQYASTYRVVSVLTVLHQDWLNGKEYKCKVSNKALPAPI
EKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHE
ALHNHYTQKSLSLSPGGSGGSGGGGSLPETGG.
Nucleotide sequence of FIX-C5-Fc-L2-P:
(SEQ ID NO: 27)
ATGCAGCGCGTCAATATGATTATGGCCGAGTCTCCAGGCCTCATTACCA
TTTGTCTGCTGGGCTACCTGCTGTCCGCAGAGTGTACCGTCTTCCTCGA
CCACGAGAACGCAAATAAGATTCTCAATAGGCCTAAGCGCTACAATTCC
GGAAAGCTCGAAGAGTTTGTCCAGGGAAATCTGGAGCGCGAGTGTATGG
AGGAAAAGTGTAGCTTTGAAGAAGCTAGAGAGGTGTTTGAGAATACTGA
GCGCACTACTGAGTTTTGGAAGCAGTATGTGGATGGCGATCAGTGCGAG
TCCAATCCATGCCTGAATGGCGGATCTTGCAAGGATGATATTAATTCTT
ACGAGTGTTGGTGCCCATTCGGCTTTGAGGGAAAGAATTGCGAACTGGA
TGTCACATGTAACATCAAGAACGGCAGATGCGAGCAGTTTTGTAAGAAC
AGCGCCGATAATAAGGTGGTGTGTAGTTGTACTGAGGGATACCGCCTCG
CAGAGAATCAGAAGTCCTGTGAGCCCGCTGTGCCATTTCCTTGTGGCCG
GGTCAGCGTCAGCCAGACTTCTAAGCTGACACGCGCCGAGACCGTCTTT
CCAGATGTGGATTATGTCAACTCTACTGAGGCCGAGACCATTCTCGATA
ATATTACTCAGTCTACACAGTCCTTTAACGACTTCACTCGGGTGGTGGG
CGGAGAGGACGCAAAGCCTGGCCAGTTTCCTTGGCAGGTCGTGCTCAAC
GGAAAGGTGGACGCATTTTGTGGCGGATCTATCGTGAATGAGAAGTGGA
TTGTGACCGCCGCTCACTGCGTCGAGACAGGCGTCAAGATTACCGTGGT
CGCTGGAGAGCACAACATTGAGGAAACAGAGCACACCGAGCAGAAGCGC
AATGTGATTCGCATCATCCCACACCACAACTACAACGCCGCTATTAATA
AGTACAACCACGACATCGCCCTGTTAGAACTAGATGAGCCTCTGGTCCT
GAATAGTTACGTCACTCCTATTTGTATCGCCGATAAAGAATACACTAAC
ATCTTTCTCAAGTTCGGCTCTGGCTATGTATCTGGATGGGGACGGGTGT
TTCACAAGGGACGGTCCGCACTCGTGCTACAATACCTCCGGGTGCCACT
GGTGGATCGGGCAACATGCCTCCGGTCTACCAAGTTTACTATATACAAT
AACATGTTTTGCGCCGGCTTTCACGAGGGCGGACGGGATAGTTGCCAGG
GCGATAGTGGAGGCCCTCATGTCACAGAGGTGGAGGGAACTAGCTTTCT
GACAGGCATTATCTCTTGGGGCGAAGAGTGCGCTATGAAGGGAAAGTAC
GGCATCTATACTAAGGTCTCGCGTTACGTCAATTGGATCAAGGAAAAGA
CAAAGCTGACAGGCGGCGGAGGGAGCGGCGGCGGAGGCTCTGGCGGCGG
CGGATCTGGCGGAGGCGGCTCTGGCGGAGGCGGATCTGTCGCTCCACCA
CCTGCACTCCCCGCCGTGGCTCCTCCACCTGCTTTGCCCGCTGGATCCG
TCGCTCCACCACCTGCACTCCCCGCCGTGGCTCCTCCACCTGCTTTGCC
CGCTGATAAGACCCACACATGTCCACCTTGCCCAGCACCTGAGCTGCTG
GGCGGACCTAGCGTGTTTCTGTTCCCACCAAAGCCAAAGGACACCCTCA
TGATTAGTAGAACCCCAGAGGTGACTTGCGTCGTGGTCGATGTCAGTCA
CGAGGACCCCGAGGTGAAGTTTAATTGGTATGTAGATGGCGTCGAGGTG
CACAACGCTAAGACCAAGCCACGCGAAGAGCAGTACGCATCTACTTACC
GGGTCGTCTCTGTGCTGACTGTGCTGCACCAGGATTGGCTCAATGGCAA
AGAGTACAAGTGTAAGGTCAGTAATAAGGCCCTCCCCGCACCTATTGAA
AAGACTATTAGCAAGGCAAAGGGCCAGCCTAGGGAGCCCCAGGTCTACA
CCCTGCCCCCTTCCCGCGATGAGCTGACTAAGAATCAGGTCTCTCTCAC
TTGCCTCGTGAAGGGCTTTTACCCATCCGACATTGCCGTGGAGTGGGAG
TCCAACGGACAGCCAGAGAATAACTACAAGACAACTCCTCCCGTGCTCG
ATAGCGACGGTTCATTCTTCCTGTACTCCAAGCTCACCGTTGATAAGTC
TCGGTGGCAGCAGGGAAATGTGTTCTCTTGTTCCGTCATGCACGAGGCA
CTGCACAATCACTACACCCAGAAGTCCCTGTCCCTCAGCCCTGGCGGCA
GCGGCGGATCTGGAGGCGGCGGATCTCTCCCAGAGACCGGCGGATAA.

27).

### b6) Construction of pFRL-rPS-FIX-GS-Fc-L2-P

The construction method of the bicistronic expression vector pFRL-rPS-FIX-GS-Fc-L2-P co-expressing FIX-GS-Fc-L2-P/Furin is the same as that of pFRL-rPS-FIX-C2-Fc-L2-P. The primers for PCR amplification of the Fc-L2-P gene include forward primer F8: 5′-GATTTGGATCCGATAAGACCCACACATGTCCACCTTG-3′ (SEQ ID

NO: 41) and reverse primer R5: 5′-TAACC GGAATTCATTATCCGCCGGTCTCTGGGAGAGATCCGCCGCCTCCAGATCCGCCGCTGC CGCCAGGGCTGAGGGACAGGGACT-3′ (SEQ ID NO: 42).

Amino acid sequence of FIX-GS-Fc-L2-P:
(SEQ ID NO: 28)
MQRVNMIMAESPGLITICLLGYLLSAECTVFLDHENANKILNRPKRYNS
GKLEEFVQGNLERECMEEKCSFEEAREVFENTERTTEFWKQYVDGDQCE
SNPCLNGGSCKDDINSYECWCPFGFEGKNCELDVTCNIKNGRCEQFCKN
SADNKVVCSCTEGYRLAENQKSCEPAVPFPCGRVSVSQTSKLTRAETVF
PDVDYVNSTEAETILDNITQSTQSENDFTRVVGGEDAKPGQFPWQVVLN
GKVDAFCGGSIVNEKWIVTAAHCVETGVKITVVAGEHNIEETEHTEQKR
NVIRIIPHEINYNAAINKYNHDIALLELDEPLVLNSYVTPICIADKEYT
NIFLKFGSGYVSGWGRVFHKGRSALVLQYLRVPLVDRATCLRSTKFTIY
NNMFCAGFHEGGRDSCQGDSGGPHVTEVEGTSFLTGIISWGEECAMKGK
YGIYTKVSRYVNWIKEKTKLTGGGGSGGGGSGGGGSGGGGSGGGGSDKT
HTCPPCPAPELLGGPSVELEPPKPKDTLMISRTPEVTCVVVDVSHEDPE
VKFNWYVDGVEVHNAKTKPREEQYASTYRVVSVLTVLHQDWLNGKEYKC
KVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQ
GNVFSCSVMHEALHNHYTQKSLSLSPGGSGGSGGGGSLPETGG.
Nucleotide sequence of FIX-GS-Fc-L2-P:
(SEQ ID NO: 29)
ATGCAGCGCGTCAATATGATTATGGCCGAGTCTCCAGGCCTCATTACCA
TTTGTCTGCTGGGCTACCTGCTGTCCGCAGAGTGTACCGTCTTCCTCGA
CCACGAGAACGCAAATAAGATTCTCAATAGGCCTAAGCGCTACAATTCC
GGAAAGCTCGAAGAGTTTGTCCAGGGAAATCTGGAGCGCGAGTGTATGG
AGGAAAAGTGTAGCTTTGAAGAAGCTAGAGAGGTGTTTGAGAATACTGA
GCGCACTACTGAGTTTTGGAAGCAGTATGTGGATGGCGATCAGTGCGAG
TCCAATCCATGCCTGAATGGCGGATCTTGCAAGGATGATATTAATTCTT
ACGAGTGTTGGTGCCCATTCGGCTTTGAGGGAAAGAATTGCGAACTGGA
TGTCACATGTAACATCAAGAACGGCAGATGCGAGCAGTTTTGTAAGAAC
AGCGCCGATAATAAGGTGGTGTGTAGTTGTACTGAGGGATACCGCCTCG
CAGAGAATCAGAAGTCCTGTGAGCCCGCTGTGCCATTTCCTTGTGGCCG
GGTCAGCGTCAGCCAGACTTCTAAGCTGACACGCGCCGAGACCGTCTTT
CCAGATGTGGATTATGTCAACTCTACTGAGGCCGAGACCATTCTCGATA
ATATTACTCAGTCTACACAGTCCTTTAACGACTTCACTCGGGTGGTGGG
CGGAGAGGACGCAAAGCCTGGCCAGTTTCCTTGGCAGGTCGTGCTCAAC
GGAAAGGTGGACGCATTTTGTGGCGGATCTATCGTGAATGAGAAGTGGA
TTGTGACCGCCGCTCACTGCGTCGAGACAGGCGTCAAGATTACCGTGGT
CGCTGGAGAGCACAACATTGAGGAAACAGAGCACACCGAGCAGAAGCGC
AATGTGATTCGCATCATCCCACACCACAACTACAACGCCGCTATTAATA
AGTACAACCACGACATCGCCCTGTTAGAACTAGATGAGCCTCTGGTCCT
GAATAGTTACGTCACTCCTATTTGTATCGCCGATAAAGAATACACTAAC
ATCTTTCTCAAGTTCGGCTCTGGCTATGTATCTGGATGGGGACGGGTGT
TTCACAAGGGACGGTCCGCACTCGTGCTACAATACCTCCGGGTGCCACT
GGTGGATCGGGCAACATGCCTCCGGTCTACCAAGTTTACTATATACAAT
AACATGTTTTGCGCCGGCTTTCACGAGGGCGGACGGGATAGTTGCCAGG
GCGATAGTGGAGGCCCTCATGTCACAGAGGTGGAGGGAACTAGCTTTCT
GACAGGCATTATCTCTTGGGGCGAAGAGTGCGCTATGAAGGGAAAGTAC
GGCATCTATACTAAGGTCTCGCGTTACGTCAATTGGATCAAGGAAAAGA
CAAAGCTGACAGGCGGCGGAGGGAGCGGCGGCGGAGGCTCTGGCGGCGG
CGGATCTGGCGGAGGCGGCTCTGGCGGAGGCGGATCCGATAAGACCCAC
ACATGTCCACCTTGCCCAGCACCTGAGCTGCTGGGCGGACCTAGCGTGT
TTCTGTTCCCACCAAAGCCAAAGGACACCCTCATGATTAGTAGAACCCC
AGAGGTGACTTGCGTCGTGGTCGATGTCAGTCACGAGGACCCCGAGGTG
AAGTTTAATTGGTATGTAGATGGCGTCGAGGTGCACAACGCTAAGACCA
AGCCACGCGAAGAGCAGTACGCATCTACTTACCGGGTCGTCTCTGTGCT
GACTGTGCTGCACCAGGATTGGCTCAATGGCAAAGAGTACAAGTGTAAG
GTCAGTAATAAGGCCCTCCCCGCACCTATTGAAAAGACTATTAGCAAGG
CAAAGGGCCAGCCTAGGGAGCCCCAGGTCTACACCCTGCCCCCTTCCCG
CGATGAGCTGACTAAGAATCAGGTCTCTCTCACTTGCCTCGTGAAGGGC
TTTTACCCATCCGACATTGCCGTGGAGTGGGAGTCCAACGGACAGCCAG
AGAATAACTACAAGACAACTCCTCCCGTGCTCGATAGCGACGGTTCATT
CTTCCTGTACTCCAAGCTCACCGTTGATAAGTCTCGGTGGCAGCAGGGA
AATGTGTTCTCTTGTTCCGTCATGCACGAGGCACTGCACAATCACTACA
CCCAGAAGTCCCTGTCCCTCAGCCCTGGCGGCAGCGGCGGATCTGGAGG
CGGCGGATCTCTCCCAGAGACCGGCGGATAA.

The structure diagram of the above FIX-L1-Fc-L2-P/Furin bicistronic expression vectors is shown in FIG. 1A.

### Construction of GGCX and VKORC1 Co-Expressing Vector

FIX is a vitamin K (VK)-dependent glycoprotein, and with the help of VK, the glutamate residue in its N-terminal Gla domain is carboxylated to form γ-carboxyglutamate, which gives FIX blood coagulation activity. During Gla modification, γ-glutamyl carboxylase (GGCX) and vitamin K epoxide reductase complex subunit 1 (VKORC 1) are two key enzymes. Therefore, the present disclosure constructed the plasmid pcDNA3.1-Hyg-VKGC to increase the expression level of functional FIX by co-transfection the plasmid with FIX. The pcDNA3.1-Hyg-VKGC plasmid structure is shown in FIG. 1B, and the construction method is as follows: gamma glutamyl carboxylase (GGCX), vitamin K epoxide reductase complex subunit 1 (VKORC1) and internal ribosome entry site (IRES) DNA were synthesized by full-gene synthesis; the IRES was cloned into the vector pcDNA3.1(+)/hygro through the Nhel/Xhol double restriction site to construct the plasmid p3.1hyg-IRES; then, through different double restriction sites, VKORC1 gene (by NheI/BamHI) and GGCX gene (by NotI/XhoI) were cloned into p3.1hyg-IRES to construct the GGCX and VKORC1 co-expressing vector pcDNA3.1-Hyg-VKGC.

### 2. Construction of Stable Cell Lines

Each type of plasmids expressing the FIX fusion protein constructed above was transfected into the host cell CHO DG44 by electrotransfection. For electroporation, 1×10⁷ cells, 40 μg of FIX-L1-Fc-L2-P/Furin co-expressing plasmid and 20 μg of pcDNA3.1-Hyg-VKGC expression plasmid were gently mixed and added to the electroporation cuvettes (Bio-Rad, 4 mm). Bio-Rad (GENE PULSER XCELL) electroporator was used and the electroporation parameters were set as follows:

| | Pulse length | Number | Pulse interval | Cuvette |
|---|---|---|---|---|
| Voltage(V) | (ms) | of Pulses | (sec) | (mm) |
| 290 | 20 | 1 | 0 | 4 |

After electroporation, the cells were transferred to recovery medium. After 24 hours of culture, the cells were seeded in fifteen 96-well plates, and the medium was changed to screening medium containing Opti-MEM I+50 nM MTX. After the cells were cultured for 4 to 5 days, medium containing 50 nM MTX was supplemented 2 to 3 times. When the cell confluence reached 50% or more, the clones were screened for high expression of target protein by Dot Blotting method, and the antibody used was mouse anti-human IgG Fc. The selected clones with relatively high expression levels were sequentially transferred to 24-well plates, 6-well plates, cell culture dishes and cell culture shake flasks for expansion.

In order to increase the yield of fusion protein, the cells were cultured under pressure with increasing MTX concentration. In this way, through the inhibition of DHFR gene by MTX, the co-amplification of DHFR gene and fusion protein gene was realized.

### 3. Production of Fusion Protein

### 3.1 Cell Thawing and Passaging

Cells were thawed and cultured in culture flasks, and after the density increased, the cells were transferred to shake flasks containing CD DG44 Medium+5 mg/L VK for culture and passage. Passage was performed when the cell density reached the range of 2.5-3.5×10⁶ cells/mL, and the passage density was between 0.8-1.0×10⁶ cells/mL. When cell seeds were expanded to 1.8-2 L and the density reached about 3.5×10⁶ cells/mL, the cells were cultured in tanks.

### 3.2 Fermentation

A 15 L bioreactor (Applikon, Biobundle 15 L) was used for fermentation, the inoculation volume was 8 L, and the cell density was 1.0×10⁶ cells/mL. The culture parameters were set as follows: 1) pH 6.8-7.4; 2) dissolved oxygen (DO) 40%; 3) oxygen supply mode: large bubble ventilation; 4) cooling strategy: culturing at 37° C. in the early stage, and cooling to 33° C. on the 5th day; 5) rotation speed: 80-140 rpm; 6) feeding strategy: feeding on the fourth day at an amount of 2%, 3%, 4%, 3%, 2%, 2%, 2%, 2%, and 2%, and the total feeding volume was 24%, and the cells were counted with a cell counter every day; 7) harvest: after culturing for about 13 days, harvesting the supernatant by centrifugation at 3000 g for 10 min.

3.3 Sample Detection and Purification

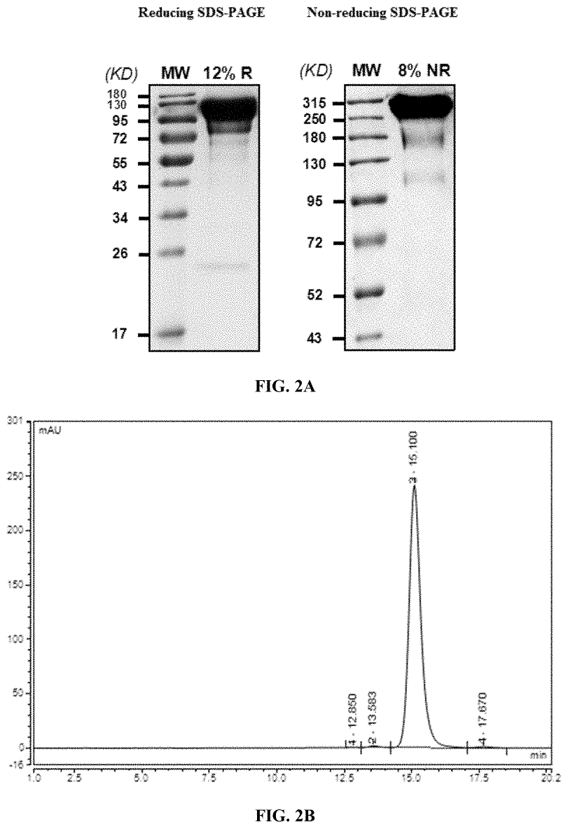

The supernatant of the fermentation broth was purified in three steps: affinity chromatography, mixed-mode chromatography and anion exchange chromatography. The purified fusion protein was detected by SDS-PAGE and SEC-HPLC. As shown in FIG. 2A, SDS-PAGE results show that the size of the interest proteins were basically the same as the theoretical molecular weight.

SEC-HPLC was performed on Agilent Advancebio SEC 300A, 2.7 7.8*300 mm column, and gradient elution was performed using 50 mM Tris+150 mM NaCl, pH 7.2 eluent, wherein the column temperature was 30° C., flow rate was 0.5 mL/min, sample injector temperature was 4.0° C., and sample injector volume was 30 μg. FIG. 2B shows the purification results using FIX-C1-Fc-L2-P protein as an example, for which the retention time was 15.1 min, and the SEC purity after three-step purification was 95.0%.

### Example 2 In Vitro Activity Detection of FIX-L1-Fc-L2-P and Comparison

In this example, the in vitro activity of FIX-C1-Fc-L2-P, FIX-C2-Fc-L2-P, FIX-C3-Fc-L2-P, FIX-C4-Fc-L2-P and FIX-C5-Fc-L2-P prepared in Example 1 were tested and compared with the in vitro activity of FIX-GS-Fc-L2-P devoid of a rigid unit in the first linker L1.

The activity of human coagulation factor IX (hFIX) was measured by the one-stage method based on activated partial thromboplastin time (APTT), where ellagic acid was used as the activator, and the 8^{th} International Standard 2009 FIX Concentrate (the standard for WHO FIX activity) was used for APTT determination. The logarithms of both the activity of the known standard and the measured APTT time were used for linear fitting to plot the standard curve. The sample to be tested was diluted to fit the range of the standard curve, and mixed with FIX-deficient plasma to measure the APTT value. The biological activity of the sample to be tested was calculated based on the standard curve.

The aliquoted WHO FIX active standard or sample was equilibrated to room temperature, then 10 μL of which was pipetted and diluted to about 1 IU/ml with 5% FIX-deficient plasma, and then was diluted 10 times and 20 times (and further diluted 40 times and 80 times for the active standard) with 5% FIX-deficient plasma and vortexed to mix well. The diluted standard or sample was placed on the analysis sample rack of the automatic coagulometer, and the reagents used were placed in the corresponding reagent positions. Then the corresponding measurement program on the automatic coagulation analyzer was employed to determine the coagulation time, and the measurement was repeated twice for each dilution. The logarithm of the standard activity was served as the abscissa and the logarithm of the coagulation time as the ordinate to plot the standard curve. The measured coagulation time of the diluted test product was substituted into the standard curve to calculate the activity result, which was then multiplied by the dilution factor to obtain the corresponding activity.

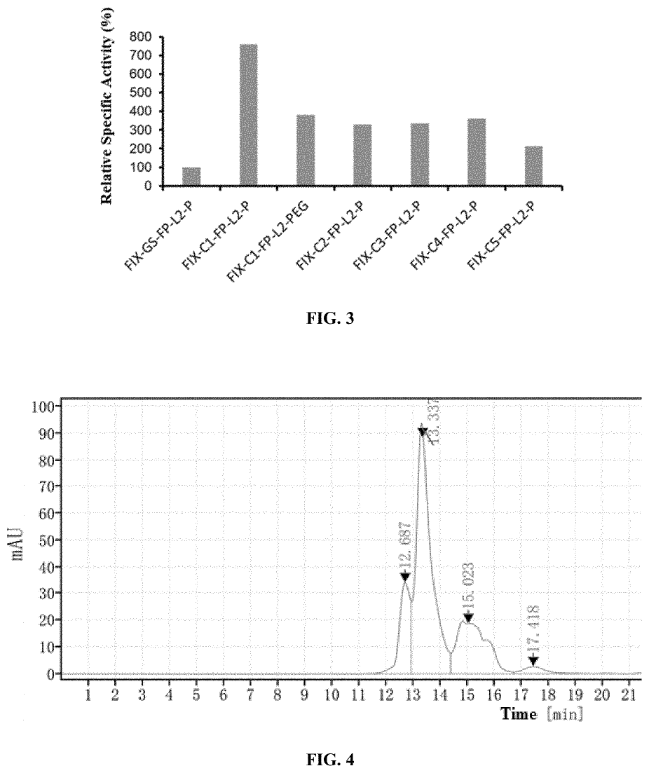

It was unexpectedly found that, as shown in FIG. 3, the specific activities of the FIX-C1-Fc-L2-P, FIX-C2-Fc-L2-P, FIX-C3-Fc-L2-P, FIX-C4-Fc-L2-P and FIX-C5-Fc-L2-P molecules in which the first linker L1 contained a rigid unit were much higher than that of the FIX-GS-Fc-L2-P in which the first linker L1 was devoid of a rigid unit, indicating that the first linker L1 containing the rigid unit can significantly increase the specific activity of the molecule.

In addition, unexpectedly, among all the fusion proteins containing the rigid unit in L1, FIX-C1-Fc-L2-P had significantly higher activity, which was nearly double or above than other fusion proteins in terms of specific activity (FIG. 3). Therefore, in the subsequent examples, FIX-C1-Fc-L2-P was selected for in-depth study.

### Example 3 Preparation of PEG-conjugated Fusion Protein FIX-C1-Fc-L2-PEG

FIX-C1-Fc-L2-P protein contains LPETGG at C-terminus, which can be specifically recognized by Sortase A. Under the action of Sortase A, the amide bond between T and G was cut, and T reacted with the sulfhydryl group of C at Sortase position 184 to form a thioester bond intermediate, which was then attacked by GGGAA-PEG with poly-Gly at the N-terminal. Finally, PEG of a specific molecular weight can be linked to the C-terminal of the protein.

PEGlation (pegylation) reaction system: 40KD-GGGAA-PEG was dissolved in buffer and adjusted to pH 7.5. Then the protein and 40KD-GGGAA-PEG were added to the 50 mM Tris 150 mM NaCl pH 7.5 buffer system at a feeding ratio of 1:20, and then Sortase A and 10 mM CaCl₂ were added. After 30 minutes, EDTA was added to terminate the reaction.

The conjugation rate of the conjugate (FIX-C1-Fc-L2-PEG) was tested by SEC-HPLC, which was performed on TOSOH TSKgel UItraSW Aggregate 7.8*300 mm 3 μm column, and gradient elution was performed using 50 mM Tris+150 mM NaCl, pH 7.2 elution buffer, where the column temperature was 25° C., flow rate was 0.5 mL/min, sample injector temperature was 4.0° C., sample injector volume was 50 the retention time of the conjugated protein was 12-14.5 min, and the retention time of the substrate was 15 min. FIG. 4 shows the detection results of FIX-C1-Fc-L2-PEG, wherein the proportion of PEG-conjugated product reached 70% at 30 min.

### Purification and Detection of PEG-Conjugated Product

The PEG-conjugated products were subjected to anion exchange chromatography to remove unreacted GGGAA-PEG, Sortase A and unconjugated substrate (FIX-C1-Fc-L2-P), and the residual amount of unconjugated substrate was controlled below 3%.

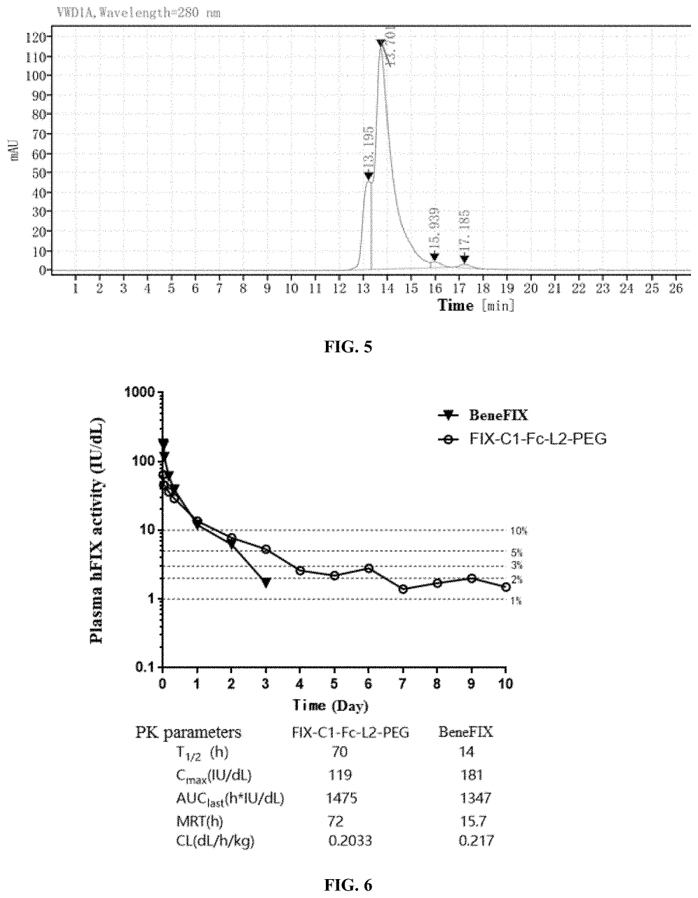

The conjugated samples were separated by anion exchange chromatography, and peaks were collected at different stages. The samples collected at each peak were detected by SDS-HPLC and SEC, and then the samples with a ratio of unconjugated substrate less than 5% were combined to detect the purity by SEC. The SEC detection result of FIX-C1-Fc-L2-PEG is shown in FIG. 5, wherein the SEC purity of the PEGylated product was 97.8%. Among them, the double-conjugated product accounted for 15.7%, with a retention time of 13.20 min, the single-conjugated product accounted for 82.2% with a retention time of 13.70 min, and the unreacted substrate FIX-C1-Fc-L2-P accounted for 1.24% with a retention time of 15.94 min.

### Example 4 FIX-C1-Fc-L2-PEG Conjugate Maintaining Excellent In Vitro Activity

This example further tested the in vitro activity of the FIX-C1-Fc-L2-PEG conjugate. The activity of human coagulation factor IX (hFIX) was measured by the one-stage method based on activated partial thromboplastin time (APTT), where ellagic acid was used as the activator, and the 8th International Standard 2009 FIX Concentrate (the standard for WHO FIX activity) was used for APTT determination. The logarithms of the activity of the known standard and the measured APTT time were used for linear fitting to plot the standard curve. The sample to be tested was diluted to fit the range of the standard curve, and mixed with FIX-deficient plasma to measure the APTT value. The biological activity of the sample to be tested was calculated based on the standard curve.

The aliquoted WHO FIX active standard or sample was equilibrated to room temperature, then 10 μl of which was pipetted and diluted to about 1 IU/ml with 5% FIX-deficient plasma, and then was diluted 10 times and 20 times (and further diluted 40 times and 80 times for the active standard) with 5% FIX-deficient plasma and vortexed to mix well. The diluted standard or sample was placed on the analysis sample rack of the automatic coagulometer, and the reagents used were placed in the corresponding reagent positions. Then the corresponding measurement program on the automatic coagulation analyzer was employed to determine the coagulation time, and the measurement was repeated twice for each dilution. The logarithm of the standard activity was served as the abscissa and the logarithm of the coagulation time as the ordinate to plot the standard curve. The measured coagulation time of the diluted test product was substituted into the standard curve to calculate the activity result, which was then multiplied by the dilution factor to obtain the corresponding activity.

As shown in FIG. 3, the specific activity of the conjugate FIX-C1-Fc-L2-PEG was lower than that of FIX-C1-Fc-L2-P, which reflects that PEG conjugation will reduce the in vitro specific activity of the molecule to a certain extent, and the above results are consistent with the common understanding in the field. However, it is also worth noting that the in vitro activity of FIX-C1-Fc-L2-PEG was still maintained at a relatively high level, not only much higher than that of FIX-GS-Fc-L2-P without PEG conjugation, but also comparable to that of other FIX-L1-Fc-L2-P fusion proteins without PEG conjugation, except for FIX-C1-Fc-L2-P.

### Example 5 FIX-C1-Fc-L2-PEG Showing a Significantly Extended Half-Life In Vivo

This example further tested the activity of the FIX-C1-Fc-L2-PEG conjugate in mice. Specifically, FIX-C1-Fc-L2-PEG was administered to HB mice, and blood sample was collected from the orbit at different time points. Then the plasma was separated for detecting the hFIX activity in the plasma by the one-stage method, and the pharmacokinetics parameters were calculated by the software WinNonlin 5.2.1.

The experimental animals used in this example are shown in the following table:

| Name | Gender | Grade | Source |
|---|---|---|---|
| HB | ♂ and ♀ | SPF | Shanghai Model |
| | | | Organisms Center, Inc. |

The test samples used in this example are as follows:

| | | Concentration |
|---|---|---|
| Name | Batch No. | (IU/ml) |
| FIX-C1-Fc-L2-PEG | 20181226 | 180 |
| BeneFIX (recombinant | N64048 | 50 |
| human coagulation | | |
| factor IX injection) | | |

This experiment was divided into 2 groups. The control drug BeneFIX and the FIX-C1-Fc-L2-PEG conjugate sample were administered through the tail vein at 300 IU/kg. The blood collection time points were: 1) BeneFIX: 0.083, 0.5, 1, 4, 8, 24, 48, and 72 h; 2) FIX-C1-Fc-L2-PEG: 0.083, 0.5, 1, 4, 8, 24, 48, 72, 96, 120, 144, 168, 192, 216, and 240 h. For each blood collection time point in each group, 5 HB animals (2 males, 3 females) were subjected to orbital blood collection and hFIX activity detection.

As shown in FIG. 6, BeneFIX can maintain plasma hFIX level at above 5% for 2 days after administration, but only 1%-2% plasma hFIX level 3 days after administration; while FIX-C1-Fc-L2-PEG still maintained plasma hFIX level at above 5% for 3 days after administration, and plasma hFIX level above 2% 6 days after administration, especially plasma hFIX level can still be maintained at 1%-2% 10 days after administration. The experimental results show that the elimination phase half-life (T1/2) of FIX-C1-Fc-L2-PEG reaches 70h, much greater than the T1/2 of the control drug BeneFIX (14h; consistent with the half-life data reported in literature) and the T1/2 of Alprolix (46h) reported in literature (Peters et al. Prolonged activity of factor IX as a monomeric Fc fusion protein. Blood 2010; 115(10): 2057-64.). The maximum blood drug concentration (Cmax) was 119 IU/dL, the drug-time area under the curve (AUClast) was 1475 h*IU/dL, the clearance rate (CL) was 0.2033 dL/h/kg, and the mean residence time (MRT) was 72 h, achieving excellent pharmacodynamic effect. It can be seen that the PEG site-directed conjugate prepared by fusing hFIX with Fc significantly increases the half-life of the hFIX molecule, and has significantly better long-acting effects in vivo than existing drugs, which has good application prospects.

1-8. (canceled)

9. A conjugate comprising a fusion protein of coagulation factor IX (FIX) and a hydrophilic polymer, wherein the hydrophilic polymer is attached to the end of the fusion protein;
the fusion protein comprises a coagulation factor IX active moiety and a fusion partner (FP),
the coagulation factor IX active moiety is full-length or truncated human coagulation factor IX and the full-length or truncated human coagulation factor IX may contain one or more amino acid mutations, provided that it still retains the FIX activity; and
the fusion partner is selected from the group consisting of full-length, truncated and variant form of immunoglobulin Fc fragment, albumin, transferrin and XTEN; and
the hydrophilic polymer is selected from polysaccharide and polyalkylene glycol.

10. The conjugate according to claim 9, wherein the hydrophilic polymer is selected from polypropylene glycol and polyethylene glycol.

11. A method for producing the conjugate according to claim 9, comprising contacting a fusion protein of coagulation factor IX (FIX) with Sortase and a hydrophilic polymer, wherein one end of the hydrophilic polymer has an amino group that can be amidated by Sortase.

12. The method according to claim 11, wherein the N-terminus of the hydrophilic polymer contains poly-Gly.

13. A pharmaceutical composition comprising an effective amount of conjugate according to claim 9, and optionally a pharmaceutically acceptable carrier.

14. (canceled)

15. The pharmaceutical composition according to claim 13, wherein the pharmaceutical composition is in a form of liquid preparation or lyophilized preparation.

16. (canceled)

17. A method for preventing and/or treating a hemorrhagic disease, comprising administering the conjugate according to claim 9 to a subject in need thereof.

18. A kit comprising the conjugate according to claim 9 and optionally instructions for use.

19. (canceled)

20. (canceled)

21. The conjugate according to claim 10, wherein the molecular weight of the polyalkylene glycol is 5 to 40 kDa.

22. The conjugate according to claim 9, wherein the coagulation factor IX active moiety comprises an amino acid sequence set forth in SEQ ID NO: 1, or an amino acid sequence having at least 85%, 90%, 95% or higher identity to the amino acid sequence shown in SEQ ID NO: 1.

23. The conjugate according to claim 9, wherein the coagulation factor IX active moiety and the fusion partner are linked directly or through a first linker L1, the first linker L1 comprises a flexible peptide and/or a rigid unit,
the flexible peptide is (GS)ₘ(GGS)ₙ(GGGS)ₒ(GGGGS)ₚ, where m, n, o and p are independently selected from integers from 0 to 50, and m, n, o and p are not all 0; and
the rigid unit comprises an amino acid sequence selected from the group consisting of:
(SEQ ID NO: 3)
VAPPPALPAPVRLPGPA,
(SEQ ID NO: 4)
VAPPPALPAVAPPPALPA,
(SEQ ID NO: 5)
VAPPPALPAVAPPPALPAVAPPPALPAPVRLPGPA,
(SEQ ID NO: 6)
VAPPPALPAPVRLPGPAVAPPPALPAVAPPPALPA,
and
(SEQ ID NO: 7)
VAPPPALPAVAPPPALPAGSVAPPPALPAVAPPPALPA.

24. The conjugate according to claim 23, wherein the first linker L1 is selected from the group consisting of:
(SEQ ID NO: 8)
GGGGSGGGGSGGGGSGGGGSGGGGSVAPPPALPAPVRLPGPA,
(SEQ ID NO: 9)
GGGGSGGGGSGGGGSGGGGSGGGGSVAPPPALPAVAPPPALPA,
(SEQ ID NO: 10)
GGGGSGGGGSGGGGSGGGGSGGGGSVAPPPALPAVAPPPALPAVAPPPAL
PAPVRLPGPA,
(SEQ ID NO: 11)
GGGGSGGGGSGGGGSGGGGSGGGGSVAPPPALPAPVRLPGPAVAPPPALP
AVAPPPALPA,
and
(SEQ ID NO: 12)
GGGGSGGGGSGGGGSGGGGSGGGGSVAPPPALPAVAPPPALPAGSVAPPP
ALPAVAPPPALPA.

25. The conjugate according to claim 9, wherein the fusion partner is Fc fragment derived from human immunoglobulin Fc fragment.

26. The conjugate according to claim 25, wherein the fusion partner is IgG Fc fragment.

27. The conjugate according to claim 9, wherein the fusion partner comprises a second linker L2: (GS)_{w}(GGS)ₓ(GGGS)_{y}(GGGGS)_{z}, where w, x, y and z are independently selected from integers from 0 to 50 and w, x, y and z are not all 0.

28. The conjugate according to claim 27, wherein the second linker L2 is selected from the group consisting of GGGGS (SEQ ID NO: 14), GGGGSGGGGS (SEQ ID NO: 15) and GSGGSGGGGS (SEQ ID NO: 16).

29. The conjugate according to claim 9, wherein the fusion protein comprises the sequence selected from the group consisting of SEQ ID NO: 18, 20, 22, 24, 26 and 28.

30. The conjugate according to claim 9, wherein the fusion protein is in a form of a monomer or a dimer.

31. The method according to claim 17, wherein the hemorrhagic disease is selected from hemorrhagic diseases in patients with congenital or acquired deficiency of FIX, and spontaneous or surgical bleeding in patients with hemophilia B.
